(19) United States
(12) Patent Application Publication
(10) Pub. No.: US 2024/0138770 A1
(43) Pub. Date: May 2, 2024

(54) SYSTEM AND METHOD FOR DETECTING SENSOR COMPRESSION OF CONTINUOUS GLUCOSE MONITORING (CGM) SENSORS

(71) Applicant: UNIVERSITY OF VIRGINIA PATENT FOUNDATION, Charlottesville, VA (US)

(72) Inventors: Boris P. KOVATCHEV, Charlottesville, VA (US); Chiara FABRIS, Charlottesville, VA (US); Marcela MOSCOSO-VASQUEZ, Charlottesville, VA (US)

(73) Assignee: UNIVERSITY OF VIRGINIA PATENT FOUNDATION, Charlottesville, VA (US)

(21) Appl. No.: 18/500,403

(22) Filed: Nov. 2, 2023

Related U.S. Application Data
(60) Provisional application No. 63/421,883, filed on Nov. 2, 2022.

(51) Int. Cl. A61B 5/00; A61B 5/145

(52) U.S. Cl. CPC A61B 5/7214; A61B 5/1451; A61B 5/14532; A61B 5/4839; A61B 5/6849; A61B 5/6885; A61B 5/746; A61B 2560/0475

(57) ABSTRACT

Embodiments can relate to systems and methods for automatically detecting sensor compression in continuous glucose monitoring in real time. A system may include at least one sensor and at least one processor in communication with the at least one sensor. The at least one processor is programmed or configured to cause the processor to retrieve first measurement data including at least one time series of blood glucose (BG) measurements, the at least one time series being measured by the at least one sensor while not subject to compression; receive, from the at least one sensor, second measurement data including at least one BG measurement; determine a clearance value between BG measurements based on the first measurement data and the second measurement data; and generate a signal output indicating that the at least one sensor is subject to compression based on the clearance value between BG measurements exceeding a predefined threshold.

### CROSS-REFERENCE TO RELATED APPLICATIONS

This patent application is related to and claims the benefit of priority to U.S. provisional patent application No. 63/421,883, filed on Nov. 2, 2022, the entire contents of which is incorporated by reference.

### FIELD

An aspect of embodiments generally relates to medicine and medical devices, as used for monitoring of blood sugar levels in the treatment of diabetes mellitus and other metabolic disorders, including but not limited to type 1 and type 2 diabetes, type 2 (T1D, T2D), latent autoimmune diabetes in adults (LADA), postprandial or reactive hyperglycemia, or insulin resistance. Embodiments relate to systems and methods for detecting that a continuous glucose monitoring (CGM) sensor is subject to compression and for detection of compression artifacts present in blood glucose (BG) measurements.

### BACKGROUND INFORMATION

Past advancements in CGM devices have contributed to the treatment of diabetes and led to some contemporary closed-loop control systems (e.g., artificial pancreas). Despite advances in CGM, CGM sensors may be vulnerable to compression artifacts (e.g., Pressure Induced Sensor Attenuations (PISAs)). Compression artifacts may occur in measured data when a sensor is pressed (e.g., subject to compression) while collecting measurements. For example, compression artifacts may occur when a subject having a sensor attached sleeps on an area (e.g., arm) where the sensor is inserted. Compression artifacts may be characterized by a rapid drop in magnitude of sensor measurements (e.g., a low reading), followed by an eventual recovery of sensor measurements (e.g., a normal reading). Such drops in sensor measurements can result in false hypoglycemia alarms, insulin shutoff in low glucose suspend or closed-loop systems, and other effects negatively impacting treatment of diabetes. Reliable methods to detect and/or anticipate compression artifacts do not exist. Prediction of compression artifacts and thereby methods to prevent compression artifacts have not been previously developed.

### SUMMARY

An exemplary embodiment can relate to a system for automatically detecting sensor compression in CGM in real time. The system may include at least one sensor. The system may also include at least one processor in communication with the at least one sensor. The at least one processor may execute program code. The at least one processor may be programmed or configured to cause the processor to retrieve first measurement data including at least one time series of BG measurements. The at least one time series may be measured by the at least one sensor while not subject to compression. The at least one processor may be programmed or configured to cause the processor to receive, from the at least one sensor, second measurement data including at least one BG measurement. The at least one BG measurement may be measured by the at least one sensor. The at least one processor may be programmed or configured to cause the processor to determine a clearance value between BG measurements based on the first measurement data and the second measurement data. The at least one processor may be programmed or configured to cause the processor to generate a signal output indicating that the at least one sensor is subject to compression based on the clearance value between BG measurements exceeding a predefined threshold.

An exemplary embodiment can relate to a system for automatically detecting end of sensor compression in continuous glucose monitoring in real time. The system may include at least one sensor. The system may also include at least one processor in communication with the at least one sensor. The at least one processor may execute program code. The at least one processor may be programmed or configured to cause the processor to receive, from the at least one sensor, first measurement data including at least one time series of BG measurements. The at least one time series of BG measurements may be measured by the at least one sensor while subject to compression. The at least one processor may be programmed or configured to cause the processor to receive, from the at least one sensor, second measurement data including at least one BG measurement. The at least one BG measurement may be measured by the at least one sensor after the at least one time series of BG measurements has been measured by the at least one sensor. The at least one processor may be programmed or configured to cause the processor to determine a clearance value between BG measurements based on the first measurement data and the second measurement data. The at least one processor may be programmed or configured to cause the processor to generate a signal output indicating that the at least one sensor is no longer subject to compression based on the clearance value between BG measurements being less than a predefined threshold.

An exemplary embodiment can relate to a computer-implemented method for accurately detecting sensor compression in continuous glucose monitoring. The method may include receiving first measurement data including at least one time series of BG measurements measured by a first sensor not subject to compression. The method may also include receiving second measurement data including plural BG measurement measured consecutively by a second sensor subject to compression. The method may also include determining plural clearance values between BG measurements based on the first measurement data and the second measurement data. The method may also include detecting that a third sensor is subject to compression based on a distribution of the plural clearance values.

### BRIEF DESCRIPTION OF THE DRAWINGS

Other features and advantages of the present disclosure will become more apparent upon reading the following detailed description in conjunction with the accompanying drawings, wherein like elements are designated by like numerals, and wherein:

FIG. 1 shows an exemplary system configuration for an embodiment of a system for detecting sensor compression of CGM sensors as disclosed herein;

FIG. 2 shows an exemplary method for detecting sensor compression of CGM sensors as disclosed herein;

FIG. 3 shows an exemplary system implementation for detecting sensor compression in CGM as disclosed herein;

FIG. 4 shows an exemplary system implementing embodiments for detecting sensor compression in CGM as disclosed herein;

FIG. 5 shows an exemplary plot of constant CGM sensor measurements without compressions artifacts and simulated CGM measurements representing compression artifacts as disclosed herein;

FIG. 6 shows an exemplary plot of CGM sensor measurements from a plurality of sensors along with plots of clearance values for each sensor for a time series of BG measurements as disclosed herein;

FIG. 7 shows an exemplary distribution of clearance values for a sensor under normal conditions and for a sensor subject to compression as disclosed herein;

FIG. 8 shows an exemplary plot of a time series of BG measurements for a single sensor including a compression artifact detected based on clearance values for the time series of BG measurements as disclosed herein;

FIGS. 9A and 9B shows an exemplary plot of area under the receiver operating characteristics curve and a precision-recall curve respectively for classifier models used to classify time series of BG measurements as including a compression artifact based on clearance values to detect sensor compression as disclosed herein;

FIG. 10A shows an exemplary system configuration of an exemplary computing device as disclosed herein;

FIG. 10B shows an exemplary system environment in which systems, methods, and/or computer-readable media may be implemented as disclosed herein;

FIG. 11 is a block diagram of an exemplary computing system in which systems, methods, and/or computer-readable mediums may be implemented as disclosed herein;

FIG. 12 shows an exemplary environment in which systems, methods, and/or computer-readable media may be implemented as disclosed herein; and

FIG. 13 is a block diagram of an exemplary machine in which systems, methods, and/or computer-readable media may be implemented as disclosed herein.

### DETAILED DESCRIPTION

Various embodiments provide for the ability to detect CGM sensor compression lows (e.g., compression artifacts) in real time and thereby prevent compression low effects negatively impacting the treatment of diabetes. Such negative impacts that may be prevented by various embodiments include false hypoglycemia alarms, and insulin shutoff in insulin delivery systems. Various embodiments may fully identify pressure induced sensor attenuations (PISAs) in real time to provide an indication that a PISA is occurring. Embodiments may improve accuracy of CGM sensors by detecting compression artifacts inherent with CGM devices. Embodiments may detect compression artifacts where detection of compression artifacts does not depend on normal physiological fluctuations of interstitial fluid (ISF) glucose, thus reducing detection of false positives. Embodiments improve action of continuous subcutaneous insulin infusion therapy and related systems, such as sensor-augmented pump (SAP), low glucose suspend (LGS), predictive low glucose suspend (PLGS), or automated insulin delivery (AID) (e.g., an artificial pancreas). Some embodiments achieved a receiver operating characteristics (ROC) curve having an area under the ROC curve as 96%, signifying generally good performance. Some embodiments produced a Precision-Recall (PR) curve having an average precision (AP) score of about 0.38, meaning that the weighted-average precision across all thresholds, with the recalls at these thresholds used as weights, is about 0.38. This result is typically considered a good result. Thus, embodiments provide reliable detection of compression artifacts in BG measurement data.

Various embodiments improve functioning of a computer (e.g., computer processor) to automatically detect PISAs and improve the treatment of diabetes. A computer that is not programmed or configured with aspects of some embodiments could not automatically detect PISAs in CGM sensor and diabetes treatment prior to the development of various embodiments disclosed herein. According to embodiments, CGM sensors may no longer be vulnerable to compression artifacts. Compression artifacts may be automatically detected in real time in measured data when a sensor is pressed (e.g., subject to compression) while collecting measurements. For example, embodiments may detect compression artifacts when a subject having a sensor attached sleeps on an area (e.g., arm) where the sensor is inserted. Embodiments may detect PISAs in such sensor measurements to anticipate and/or prevent false hypoglycemia alarms, insulin shutoff in low glucose suspend or closed-loop systems, and other effects that may negatively impact treatment of diabetes. Embodiments may accurately predict compression artifacts such that compression artifacts can be handled in real time when they occur to enhance treatment of diabetes.

Embodiments may continually determine, in real time, a clearance value such that clearance values can be plotted and identified over time while a CGM sensor is collecting measurements. Such determination is performed while measurement data is collected by a CGM sensor and is performed continuously to provide a holistic view over time of a sensor's status with respect to compression. Real-time, iterative determination of clearance values allows embodiments to improve the functioning of a computer such that real-time compression artifacts can be detected and handled or remediated.

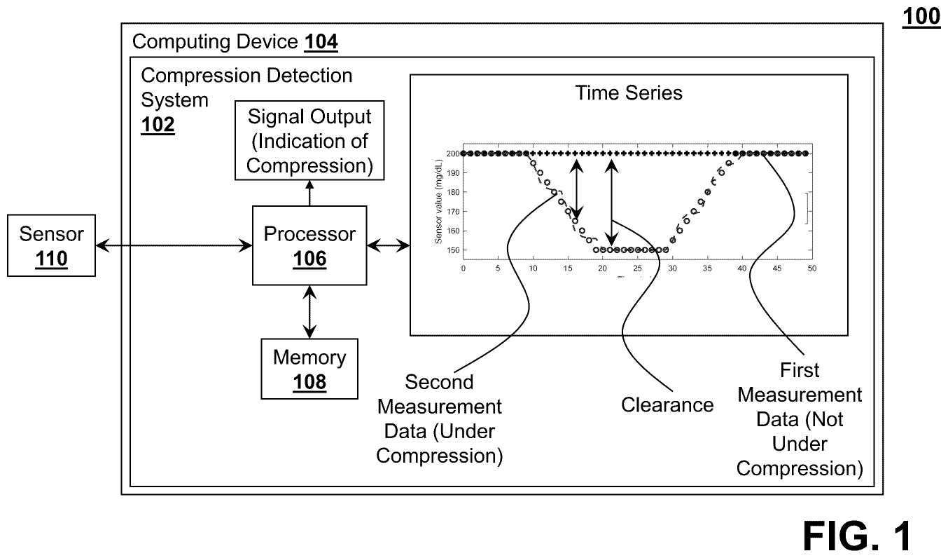

FIG. 1 shows an exemplary system configuration 100 for an embodiment of a system operable via program code (e.g., software instructions executed by a processor) for detecting sensor compression (e.g., via compression artifacts) in CGM. The various components of FIG. 1 may be implemented in and/or processed by a processor (e.g., a central processing unit (CPU)) and/or on any number of distributed processors (e.g., a distributed computing system) coupled with memory and connected via a communications network. Each of the components shown in FIG. 1 are described in the context of an exemplary embodiment.

As shown in FIG. 1, embodiments relate to a system configured for detecting sensor compression in CGM. In some embodiments, system configuration 100 may automatically detect sensor compression in CGM in real time. In some embodiments, system configuration 100 may include compression detection system 102, computing device 104, processor 106, memory 108, and sensor 110.

In some embodiments, system configuration 100 may include at least one sensor (e.g., sensor 110). The at least one sensor may measure and/or collect BG measurements associated with time stamps. The BG measurements (e.g., BG measurement data) may be in the form of a time series including plural time stamps. For example, each BG measurement measured and/or collected by the at least one sensor may be associated with exactly one time stamp, such that a collection of plural BG measurements forms a time series of BG measurements collected by the at least one sensor over time. The at least one time series of BG measurements may span various amounts of time and may be of any various lengths (e.g., number of time stamps and BG measurements in a time series). For example, at least one time series of BG measurements may include 30 minutes (e.g., in duration) of measurement data measured by at least one sensor. The at least one sensor may measure and/or collect BG measurements over time at specified time intervals (e.g., every 30 seconds, every minute, and/or the like) which may define a resolution of a time series of BG measurements. For example, plural time stamps of a time series of BG measurements may be separated by any one or more of 30 second intervals, 1 minute intervals, 2.5 minute intervals, and/or 5 minute intervals. Other time intervals may be used for collecting measurements and/or for resolution of time series.

In some embodiments, system configuration 100 may include plural sensors. For example, system configuration 100 may include at least one additional sensor (including sensor 110). The at least one processor may be programmed or configured to cause the processor to receive first measurement data including at least one time series of BG measurements from at least one additional sensor. The at least one processor may store the first measurement data collected by the additional sensor in the at least one memory device (e.g., memory 108).

In some embodiments, system configuration 100 may include at least one processor (e.g., processor 106) in communication with the at least one sensor. The at least one processor may execute program code (e.g., software instructions) for performing one or more steps of a method. In some embodiments, compression detection system 102 may include program code for one or more steps for embodiments described herein. The at least one processor may execute program code for detecting sensor compression in CGM in real time with respect to the at least one sensor collecting measurement data.

In some embodiments, at least one processor may be programmed or configured (e.g., via software instructions) to cause the processor to receive, from the at least one sensor, measurement data including at least one time series of BG measurements measured by the at least one sensor. The measurement data may be transmitted from the at least one sensor to the at least one processor in real time (e.g., with respect to the sensor collecting the measurement data). Alternatively, the measurement data may be transmitted from the at least one sensor to memory (e.g., memory 108) coupled with the at least one processor such that the at least one processor may access the measurement data at a later time with respect to when the at least one sensor collected the measurement data.

In some embodiments, the at least one time series of BG measurements may include plural time stamps. Each time stamp may be associated with a BG measurement. For example, each time stamp may represent a relative or absolute time for when the BG measurement was collected by a sensor (e.g., sensor 110). In some embodiments, the plural time stamps may be separated by any one or more of 30 second intervals, 1 minute intervals, 2.5 minute intervals, and/or 5 minute intervals.

In some embodiments, at least one processor may be programmed or configured (e.g., via software instructions) to cause the processor to retrieve first measurement data including at least one time series of BG measurements. For example, the at least one processor may retrieve first measurement data from memory coupled to the at least one processor. The first measurement data may be collected by at least one sensor and transmitted to memory for storage and/or transmitted to the at least one processor. The at least one time series of BG measurements may be measured by the at least one sensor while subject to compression or while not subject to compression. In some embodiments, the at least one processor may be programmed or configured to cause the processor to receive the first measurement data from the at least one sensor (e.g., in real time). In this way, the at least one processor may be programmed or configured to cause the processor to receive the first measurement data for immediate use and/or the at least one processor may be programmed or configured to cause the processor to retrieve the first measurement data from a storage component (e.g., memory 108) for later use after the first measurement data has been collected by the at least one sensor. In some embodiments, the at least one time series of BG measurements (e.g., first measurement data) may be measured and/or collected by the at least one sensor prior to second measurement data being measured and/or collected.

In some embodiments, at least one processor may be programmed or configured to cause the processor to receive, (e.g., from the at least one sensor), second measurement data. The second measurement data may include at least one BG measurement. The at least one BG measurement may be measured by the at least one sensor (e.g., in real time). The second measurement data may be transmitted from the at least one sensor to the at least one processor in real time (e.g., with respect to the sensor collecting the second measurement data). Alternatively, the second measurement data may be transmitted from the at least one sensor to memory (e.g., memory 108) coupled with the at least one processor such that the at least one processor may access the second measurement data at a later time with respect to when the at least one sensor collected the second measurement data. In this way, the at least one processor may be programmed or configured to cause the processor to receive the second measurement data for immediate use and/or the at least one processor may be programmed or configured to cause the processor to retrieve the second measurement data from a storage component (e.g., memory 108) for later use after the second measurement data has been collected by the at least one sensor.

In some embodiments, at least one processor, as configured to receive second measurement data including at least one BG measurement, may be programmed or configured to cause the processor to receive, from the at least one sensor, the second measurement data including consecutive BG measurements (e.g., plural BG measurements) as the BG measurements are obtained by the at least one sensor in real time.

In some embodiments, at least one processor may be programmed or configured to cause the processor to determine that the at least one time series of BG measurements is a candidate series including a compression artifact. A candidate series may include a time series of BG measurements that includes a drop time-window (e.g., a series of BG measurements decreasing in value over time). A candidate series may include a time series of BG measurements that includes one or more BG measurement values below a BG measurement threshold (e.g., a threshold value). In some embodiments, a candidate series may include a time series of BG measurements that contains one or more attributes that may indicate that the time series of BG measurements includes a compression artifact. However, in some instances, a candidate series may contain one or more attributes indicating that the time series of BG measurements includes a compression artifact, while the time series of BG measurements does not include a compression artifact. In other instances, the candidate series will include a compression artifact. Determining that the at least one time series of BG measurements is a candidate series may be a first step for finding a compression artifact in measurement data and detecting sensor compression.

In some embodiments, at least one processor may be programmed or configured to cause the processor to determine that the at least one time series of BG measurements includes a change in BG measurement values across plural time stamps (e.g., across two consecutive time stamps, or across multiple time stamps beginning from a first time stamp). The change in BG measurement values may exceed a threshold (e.g., a threshold value). For example, at least one processor may calculate a change in BG measurement values by determining a difference between a first BG measurement value and a second BG measurement value to determine the change in BG measurement values. At least one processor may then determine that the difference between the first BG measurement value and the second BG measurement value exceeds a threshold (e.g., a drop time-threshold, a rise time-threshold, and/or the like). In some embodiments, the first BG measurement value may be associated with a first time stamp and the second BG measurement value may be associated with a second time stamp, where the first time stamp and second time stamp are consecutive time stamps in the at least one time series of BG measurements. Alternatively, the first BG measurement value may be associated with a first time stamp and the second BG measurement value may be associated with a second time stamp, where the first time stamp and second time stamp are separated by one or more time stamps in the at least one time series of BG measurements.

In some embodiments, at least one processor may be programmed or configured to cause the processor to determine a clearance value between BG measurements (e.g., a clearance value between a first BG measurement value and a second BG measurement value) based on the first measurement data and the second measurement data. For example, the at least one processor may be programmed or configured to cause the processor to select a first BG measurement value from the first measurement data and select a second BG measurement value from the second measurement data. The at least one processor may be programmed or configured to cause the processor to determine the clearance value based on a difference between the first BG measurement value and the second BG measurement value. The at least one processor may be programmed or configured to cause the processor to determine the clearance value between the at least one BG measurement of the second measurement data and a first BG measurement associated with a first time stamp of the first measurement data.

In some embodiments, at least one processor, as configured to determine a clearance value between BG measurements, may be programmed or configured to cause the processor to determine each clearance value of plural clearance values between each consecutive BG measurement and each BG measurement of the first measurement data in real time as each consecutive BG measurement is received. A first BG measurement of the consecutive BG measurements may be associated with a first time stamp of the first measurement data, a second BG measurement of the consecutive BG measurements may be associated with a second time stamp, and so on for the consecutive BG measurements. In this way, each consecutive BG measurement may have a clearance value determined for the BG measurement as the BG measurement is collected by at least one sensor and received by at least one processor in real time.

In some embodiments, at least one processor may be programmed or configured to cause the processor to determine a clearance value based on a model, such as:

$$\frac{d {G}_{L S C}}{d t} = - {k}_{1} * {G}_{L S C} + {k}_{0} * {G}_{ISF}$$

where, G_{LSC} is a concentration of glucose in a local sensor compartment of the at least one sensor, G_{ISF} is a concentration of glucose of interstitial fluid, dG_{LSC}/dt is a rate of change of the concentration of glucose in the local sensor compartment, k₁ is the clearance value, and k₀ is a 1 glucose transport rate where

$${k}_{0} = \frac{1}{min} .$$

In some embodiments, the at least one processor, as configured to determine that the at least one time series of BG measurements is a candidate series, may be programmed or configured to cause the processor to determine that the at least one time series of BG measurements includes a time series sub-sequence having a drop time-window and having a rise time-window associated with the drop time-window. A time series sub-sequence may include a series of BG measurements associated with time stamps in the form of a time series including plural time stamps that is a subset of the at least one time series of BG measurements. For example, each BG measurement measured and/or collected by the at least one sensor may be associated with exactly one time stamp, such that a collection of plural BG measurements forms a time series of BG measurements collected by the at least one sensor over time. The time series of BG measurements may include one or more time series sub-sequences that may span various amounts of time and may be of any various lengths (e.g., number of time stamps and BG measurements in a time series sub-sequence) within the time series of BG measurements.

In some embodiments, the time series sub-sequence may include a sequence of time stamps corresponding to at least a portion of the drop time-window (e.g., at least one time stamp of the time series sub-sequence is within the drop time-window) and at least a portion of the rise time-window (e.g., at least one time stamp of the time series sub-sequence is within the rise time-window). In some embodiments, the time series sub-sequence may span at least 2.5 minutes in duration of BG measurements.

In some embodiments, at least one processor, as configured to determine that the at least one time series of BG measurements is a candidate series, may be programmed or configured to cause the processor to determine a drop time-window within the at least one time series based on a difference between a first BG measurement value and a second BG measurement value exceeding a drop time-threshold. The drop time-window may include a series of plural time stamps and plural BG measurements that begin at a first time stamp associated with the first BG measurement (e.g., BG measurement value) and end at a second time stamp associated with the second BG measurement (e.g., BG measurement value). In some embodiments, the first BG measurement value and the second BG measurement value may be part of the same measurement data (e.g., time series of BG measurements).

In some embodiments, at least one processor, as configured to determine that the at least one time series of BG measurements is a candidate series, may be programmed or configured to cause the processor to determine a rise time-window within the at least one time series based on a difference between a third BG measurement value and a fourth BG measurement value exceeding a rise time-threshold. The rise time-window may include a series of plural time stamps and plural BG measurements that begin at a third time stamp associated with the third BG measurement (e.g., BG measurement value) and end at a fourth time stamp associated with the fourth BG measurement (e.g., BG measurement value). A rise time-window may be associated with a drop time-window in that a rise time-window can only occur within a time series of BG measurements after at least one drop time-window has occurred.

In some embodiments, a drop-time threshold may be equal to 10 mg/dL (e.g., a BG measurement value). In some embodiments, a rise-time threshold may be equal to 6 mg/dL (e.g., a BG measurement value).

In some embodiments, at least one processor may be programmed or configured to cause the processor to generate a signal output indicating that the at least one sensor is subject to compression based on the clearance value between BG measurements. For example, the at least one processor may generate a signal output indicating that the at least one sensor is subject to compression based on the clearance value exceeding a predefined threshold (e.g., a threshold defined by a BG measurement value).

In some embodiments, at least one processor may be programmed or configured to cause the processor to generate a signal output indicating that the at least one time series of BG measurements was obtained while the at least one sensor was subject to compression. For example, the signal output may include a signal transmitted to an insulin delivery system to cause the insulin delivery system to perform an action, a signal transmitted to a display, a signal transmitted to another processor to cause the processor to perform an action, and/or the like.

In some embodiments, at least one processor, as configured to generate a signal output, may be programmed or configured to cause the processor to predict that the at least one sensor is subject to compression while the at least one sensor is obtaining a BG measurement. At least one processor may be programmed or configured to cause the processor to predict (e.g., generate a prediction) via a predefined model executed by the processor. The processor may generate a prediction in real time via outputting an indication based on receiving measurement data from the at least one sensor as the sensor is collecting the measurement data (e.g., in real time with respect to the sensor collecting the measurement data).

In some embodiments, at least one processor, as configured to generate a signal output, may be programmed or configured to cause the processor to predict, in real time via outputting an indication, that the at least one sensor is subject to compression while the at least one sensor is obtaining a BG measurement. The at least one processor, as configured to generate a signal output, may be programmed or configured to cause the processor to indicate, in real time via outputting an indication, that the at least one sensor is subject to compression while the at least one sensor is obtaining a BG measurement.

For example, at least one processor may generate a signal output based on a determination that a clearance value is outside of a normal distribution of clearance values. The normal distribution of clearance values may represent magnitudes of clearance values that are normally determined by at least one processor when the at least one sensor collected BG measurements while the at least one sensor was not subject to compression.

In some embodiments, at least one processor may generate a signal output based on a determination that a clearance value is within a distribution of clearance values for compression lows. The distribution of clearance values for compression lows may represent magnitudes of clearance values that are determined by at least one processor when the at least one sensor collected BG measurements while the at least one sensor was subject to compression. In some embodiments, the distribution of clearance values for compression lows may be determined experimentally (e.g., predetermined), using one or more sensors, where the one or more sensors are known to be collecting BG measurements while subject to compression and/or while not subject to compression.

In this way, at least one processor may generate a clearance value in real time that may be compared to a normal distribution of clearance values and a distribution of clearance values for compression lows. Based on comparing the clearance values with the distributions, the at least one processor may identify whether a time series includes a BG measurement obtained while the at least one sensor was subject to compression in real time. That is, BG measurement values may be collected and compared with previous BG measurement values (e.g., based on a delay) from the at least one sensor such that the BG measurement values used to determine the clearance value are close in time (e.g., collected by the at least one sensor within 30 seconds to 5 minutes of each other) to allows for comparison of the clearance values with the distributions in real time. In some embodiments, the at least one time series of BG measurements (e.g., first measurement data) may include at least one BG measurement extrapolated (and not measured) from the at least one time series of BG measurements. The at least one processor may then generate a signal output indicating whether the sensor is subject to compression, based on the comparison of the clearance value with the distributions.

In some embodiments, at least one processor may be programmed or configured to cause the processor to identify one or more features of the at least one time series of BG measurements. For example, the at least one processor may identify and/or extract features of the at least one time series of BG measurements for inputting into at least one machine learning model (e.g., via feature extraction techniques). At least one processor may be programmed or configured to cause the processor to input the one or more features into at least one machine learning model for classification and/or generating a signal output based on a classification of the at least one time series of BG measurements.

In some embodiments, at least one processor may be in combination with an insulin delivery system that is in communication with the at least one processor. The at least one processor may be programmed or configured to cause the processor to transmit a signal output to the insulin delivery system. The signal output may indicate that the at least one sensor is subject to compression, and the signal output may cause the insulin delivery system to perform at least one or more of initiating insulin delivery (e.g., after insulin delivery was stopped due to compression artifacts), continuing insulin delivery (e.g., after determining that the at least one sensor is subject to compression and is functioning correctly), disabling an alarm (e.g., after sensor compression caused triggering of an alarm), and/or any combination thereof.

Referring to FIG. 1, compression detection system 102 may include software instructions (e.g., program code) implemented on computing device 104. Compression detection system 102 may include memory 108 storing the software instructions. Compression detection system 102 may include processor 106 executing the software instructions to cause processor 106 to perform one or more functions. Compression detection system 102 may include sensor 110 (e.g., a CGM sensor).

Compression detection system 102 may include at least one processor 106. At least one processor 106 may generate at least one signal output based on at least one time series of BG measurement data having a compression artifact supplied as a runtime input to at least one processor 106. A signal output of at least one processor 106 may include an indication of whether the at least one time series of BG measurement data was obtained while the at least one sensor was subject to compression. The at least one time series of BG measurements may be received by computing device 104 and/or processor 106 from memory 108 and/or sensor 110. Additionally or alternatively, at least one processor 106 may generate at least one signal output (e.g., a prediction) based on training and/or testing datasets.

In some embodiments, compression detection system 102 may be implemented in a single computing device. In some embodiments, compression detection system 102 may be implemented in plural computing devices (e.g., a group of servers, such as a group of computing devices 104, and/or the like) as a distributed system such that software instructions are implemented on different computing devices. In some embodiments, compression detection system 102 may be associated with computing device 104, such that compression detection system 102 is executed on computing device 104 or a portion of compression detection system 102 is executed on computing device 104 as part of a distributed computing system where sensor 110 is not part of computing device 104. Alternatively, compression detection system 102 may include at least one computing device 104 executing software instructions and at least one sensor 110 for detecting compression artifacts and/or PISAs.

Sensor 110 may include a CGM sensor that is configured to detect and/or measure blood glucose within a subject (e.g., a patient). In some embodiments, sensor 110 may include one or more sensors. For example, sensor 110 may include a CGM sensor and a pressure sensor. In some embodiments, sensor 110 may include plural CGM sensors. Sensor 110 may be worn and/or attached to a subject on various parts of the subject's body (e.g., arm, abdomen, or the like). Sensor 110 may be configured to collect measurements (e.g., BG measurements) and transmit measurements to a processor (e.g., processor 106). Sensor 110 may be subject to compression (e.g., via a subject, or other means) while sensor 110 is collecting and/or obtaining measurements, thereby affecting an accuracy of the measurements obtained by sensor 110.

Sensor 110 may include a sampling rate that may be configured for collecting and/or obtaining measurements over time having a particular sampling resolution. For example, sensor 110 may be configured to sample measurements at 30 second intervals, 1 minute intervals, 2.5 minute intervals, 5 minute intervals, and/or the like. Sensor 110 may transmit sampled measurements to processor 106 in real time as measurements are sampled. In some embodiments, sensor 110 may transmit at least one time series of sampled measurements to processor 106 and/or memory 108 at specified time intervals. A time series may include plural BG measurements, each BG measurement associated with a time stamp. The time stamp may represent an absolute or relative time when the BG measurement was collected by sensor 110. Sensor 110 may be in communication with computing device 104 and/or processor 106 via wired (e.g., a data bus, ethernet, and/or the like) or wireless (e.g., Wi-Fi, Bluetooth, and/or the like) means and/or a communication interface.

Computing device 104 may include processor 106 (e.g., CPU) and memory 108. Processor 106 may execute software instructions (e.g., compiled program code) for compression detection system 102. In some embodiments, sensor 110 may be separate from computing device 104. Alternatively, sensor 110 may be integrated with (e.g., part of) computing device 104.

Computing device 104 may include one or more processors (e.g., processor 106) configured to execute software instructions. For example, computing device 104 may include a desktop computer, a portable computer (e.g., laptop computer, tablet computer), a workstation, a mobile device (e.g., smartphone, cellular phone, personal digital assistant, wearable device), a server, and/or other like devices. Computing device 104 may include a computing device configured to communicate with one or more other computing devices over a network. Computing device 104 may include a group of computing devices (e.g., a group of servers) and/or other like devices. In some embodiments, computing device 104 may include a data storage device. Alternatively, a data storage device may be separate from computing device 104 and may be in communication with computing device 104.

Processor 106 may be implemented in hardware, software, or a combination of hardware and software. For example, processor 106 may include a common processor (e.g., a CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed with software instructions such that the processor is configured to cause the processor to perform functions when executing the software instructions. In some embodiments, processor 106 may include plural processors implemented in a single computing device 104 (e.g., a CPU and a GPU) or processor 106 may include plural processors implemented among plural distributed computing devices 104. Processor 106 may be coupled to memory 108 via a data bus to transfer data between processor 106 and memory 108. In some embodiments, processor 106 may be coupled to sensor 110 via wired (e.g., a data bus, ethernet, and/or the like) or wireless (e.g., Wi-Fi, Bluetooth, and/or the like) means and/or communication interface.

Memory 108 may include random access memory (RAM), read-only memory (ROM), and/or another type of dynamic or static storage device (e.g., flash memory, magnetic memory, optical memory, etc.) that stores information and/or software instructions for use by processor 106. Memory 108 may include a computer-readable medium and/or storage component. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 108 from another computer-readable medium or from another device via a communication interface with computing device 104. When executed, software instructions stored in memory 108 and executed by processor 106 may cause processor 106 to perform one or more functions described herein. Embodiments described herein are not limited to any specific combination of hardware circuitry and software.

The number and arrangement of systems, hardware, and/or modules (e.g., software instructions) shown in FIG. 1 is provided as an example. There may be additional systems, hardware, and/or modules, fewer systems, hardware, and/or modules, different systems, hardware, and/or modules, or differently arranged systems, hardware, and/or modules than those shown in FIG. 1. Furthermore, two or more systems, hardware, and/or modules shown in FIG. 1 may be implemented within a single system, hardware, and/or module. A single system, hardware, and/or module shown in FIG. 1 may be implemented as multiple, distributed systems, hardware, and/or modules. Additionally or alternatively, a set of systems, a set of hardware, and/or a set of modules (e.g., one or more systems, one or more hardware devices, one or more modules) of FIG. 1 may perform one or more functions described as being performed by another set of systems, another set of hardware, or another set of modules of FIG. 1.

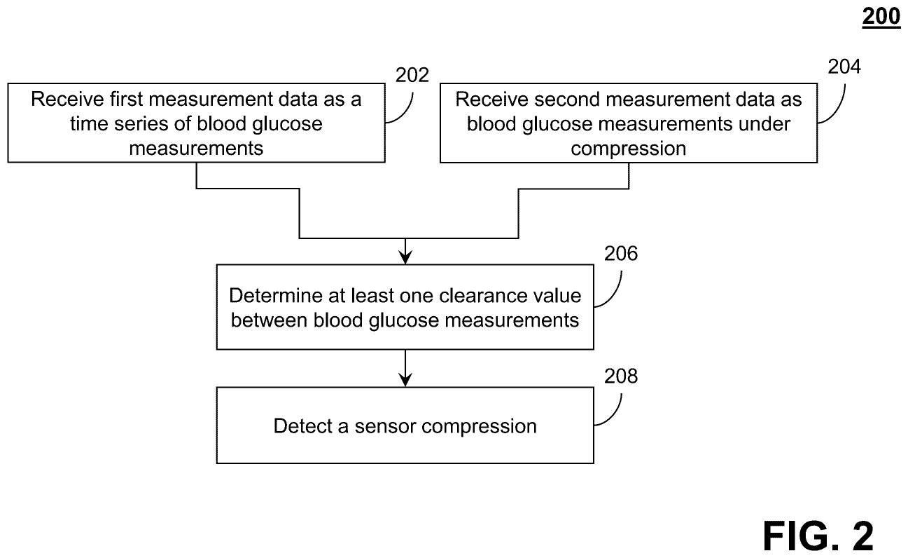

As shown in FIG. 2, embodiments relate to an exemplary method 200 for detecting sensor compression in continuous glucose monitoring as disclosed herein. Method 200 may be performed by one or more components of system configuration 100. In some embodiments, one or more of the functions described with respect to method 200 may be performed (e.g., completely, partially, etc.) by compression detection system 102 (e.g., via processor 106). In some embodiments, one or more of the steps of method 200 may be performed (e.g., completely, partially, etc.) by another system, hardware, or module or a group of systems, hardware, or modules separate from or including compression detection system 102, such as a client device and/or a separate computing device.

As shown in FIG. 2, at step 202, method 200 may include receiving first measurement data as at least one time series of BG measurements. For example, compression detection system 102 (e.g., via computing device 104 and/or processor 106) may receive at least one time series of BG measurements from sensor 110. In some embodiments, compression detection system 102 may receive the first measurement data for providing the measurement data as input to at least one processor 106 for generating a signal output. The first measurement data may include historical data (e.g., data collected at a previous time). Alternatively, the first measurement data may include real-time runtime inputs from sensor 110. In some embodiments, the first measurement data may be received from sensor 110 or another sensor. In some embodiments, the first measurement data may be retrieved from one or more storage devices, such as memory 108, when the first measurement data includes historical data. The first measurement data was collected by at least one sensor (e.g., sensor 110) while the at least one sensor was not subject to compression.

In some embodiments, the first measurement data may include plural time series of BG measurements. A time series of BG measurements may include plural time stamps. Each time stamp may be associated with a BG measurement. For example, one BG measurement collected by sensor 110 may be associated with exactly one time stamp. A time stamp may represent an absolute or relative time when the BG measurement was collected by sensor 110. Processor 106 may receive the first measurements data including a time series of BG measurements as input to processor 106. A time series of BG measurements may span various amounts of time and may be of any various lengths (e.g., number of time stamps and BG measurements). For example, at least one time series of BG measurements may include at least 30 minutes of measurement data measured by at least one sensor. In this example, the number of time stamps and BG measurements may depend on the resolution of the BG measurements, or a sample rate at which sensor 110 collects the BG measurements. In some embodiments, a time series sub-sequence may span at least 2.5 minutes in duration of BG measurements. In some embodiments, a time series sub-sequence may be longer or shorter than a 2.5 minute duration.

At step 204, method 200 may include receiving second measurement data including at least one BG measurement. For example, compression detection system 102 (e.g., via computing device 104 and/or processor 106) may receive at least one time series of BG measurements from sensor 110 that are collected while sensor 110 is under compression. In some embodiments, compression detection system 102 may receive the second measurement data for providing the measurement data as input to at least one processor 106 for generating a signal output. The second measurement data may include historical data (e.g., data collected at a previous time). Alternatively, the second measurement data may include real-time runtime inputs from sensor 110 (e.g., measurement data received by processor 106 at the same time or shortly after sensor 110 collects the measurement data). In some embodiments, the first measurement data may be received from sensor 110 or another sensor. In some embodiments, the first measurement data may be retrieved from one or more storage devices, such as memory 108, when the first measurement data includes historical data.

In some embodiments, step 202 and step 204 of method 200 may occur separately, either with step 202 occurring prior to step 204, or step 204 occurring prior to step 202. In some embodiments, step 202 may occur simultaneously with step 204.

At step 206, method 200 may include determining at least one clearance value. For example, compression detection system 102 (e.g., via computing device 104 and/or processor 106) may determine a clearance value between BG measurements based on the first measurement data and the second measurement data. A clearance value may include a value based on a model for the at least one BG measurement of the second measurement data and a first BG measurement associated with a first time stamp of the first measurement data.

At step 208, method 200 may include detecting a sensor compression. For example, compression detection system 102 may detect that at least one sensor is subject to compression based on the clearance value. In some embodiments, compression detection system 102 may detect that at least one sensor is subject to compression based on a distribution of plural clearance values.

In some embodiments, compression detection system 102 may determine that the measurement data contains a compression artifact based on determining that at least one BG measurement value is less than a compression estimate threshold. For example, one or more BG measurements (e.g., within a time series of BG measurements) may form a compression artifact in the measurement data. The one or more BG measurements may indicate an onset of sensor compression (e.g., that sensor 110 was subject to compression while collecting the one or more BG measurements).

In some embodiments, compression detection system 102 may determine that the second measurement data received from sensor 110 contains an onset of sensor compression based on determining that the measurement data (e.g., a time series sub-sequence of the measurement data) contains at least one compression artifact. A compression artifact may include a time series of BG measurements where a clearance value of each BG measurement in the time series is above or below a predefined threshold such that the clearance values are out of a normal range (e.g., outside of a band of equilibrium clearance values which may indicate a normal BG level in a subject).

In some embodiments, compression detection system 102 (e.g., via computing device 104 and/or processor 106) may identify one or more features of the at least one time series of BG measurements. Compression detection system 102 may input the one or more features into at least one machine learning model for classification and/or for generating a signal output.

Compression detection system 102 may generate a signal output indicating that the at least one BG measurement (e.g., of the second measurement data) was obtained while the at least one sensor (e.g., sensor 110) was subject to compression. In some embodiments, compression detection system 102 may generate the signal output based on determining a clearance value and determining if the clearance value is within a distribution of clearance values for compression lows.

In some embodiments, compression detection system 102 (e.g., via computing device 104 and/or processor 106), as configured to generate a signal output, may predict, in real time via outputting an indication, that the at least one sensor is subject to compression while the at least one sensor is obtaining a BG measurement. For example, compression detection system 102 may generate a signal output based on a clearance value determined by compression detection system 102. The clearance value may be determined via a model (e.g., physiological model) that models glucose diffusion between an interstitial fluid and local sensor compartment where a sensor (e.g., sensor 110) may be collecting BG measurements. The signal output (e.g., the clearance value) indicating that the at least one sensor is subject to compression may be determined using the model and measurement data that includes a BG measurement obtained while the at least one sensor was subject to compression. In this way, compression detection system 102 may use a physiological model to generate clearance values that can be used to determine that a BG measurement was obtained while the at least one sensor was subject to compression. Clearance values having a value that is within a distribution of clearance values for compression lows may indicate that the BG measurements (e.g., the BG measurements associated with the clearance values) were collected by the at least one sensor while the at least one sensor was subject to compression. Alternatively, clearance values having a value that is outside of a distribution of clearance values for compression lows (and within a normal distribution of clearance values) may indicate that the BG measurements (e.g., the BG measurements associated with the clearance values) were collected by the at least one sensor while the at least one sensor was not subject to compression.

In some embodiments, when compression detection system 102 determines the clearance value, compression detection system 102 may generate a model output of BG measurements including BG measurements (e.g., second measurement data) that are a function of glucose concentration in a local sensor compartment. Compression detection system 102 may receive a model input of BG measurements, where the model input of BG measurements represents an estimate of glucose concentration in an interstitial fluid that is associated with the local sensor compartment. In some embodiments, compression detection system 102 may determine a clearance value based on the model input, model output, and the physiological model. For example, compression detection system 102 may determine the clearance value based on the following model:

$$\frac{d {G}_{L S C}}{d t} = - {k}_{1} * {G}_{L S C} + {k}_{0} * {G}_{ISF}$$

where G_{LSC} is a glucose concentration at a local sensor compartment, G_{ISF} is a glucose concentration at an interstitial fluid associated with the local sensor compartment, k₁ is the clearance value, and k₀ is a glucose transport rate to the local sensor compartment, which is set to

$${k}_{0} = \frac{1}{min} .$$

In some embodiments, the clearance value may increase (e.g., may have a higher value) when a difference between G_{ISF} and G_{LSC} is detected. In some embodiments, when compression detection system 102 uses the physiological model, the clearance value may not depend upon G_{ISF}. In this way, clearance values may be dependent on differences between G_{ISF} and G_{LSC} and may be independent from fluctuations in glucose concentration. This may ensure that detection of compression lows does not depend on any normal physiological fluctuations and may be only dependent on compression of a sensor.

In some embodiments, real time may include an instant in time with respect to the occurrence of an event (e.g., real time with respect to collection of measurement data) where a response (e.g., generating a signal output) may occur within a specified time, generally a relatively short time (e.g., within seconds or less) of the event occurring. For example, real time may refer to an instant in time where a signal output is generated by compression detection system 102 concurrent with or shortly after (e.g., within milliseconds or seconds) the collection of measurement data by sensor 110 (or another sensor). As a further example, a real-time (e.g., runtime) signal output may be generated with respect to collecting and/or receiving a time series of BG measurements or at least one BG measurement concurrent with or shortly after compression detection system 102 receives the time series of BG measurements or the at least one BG measurement and/or concurrent with or shortly after compression detection system 102 determines a clearance value.

In some embodiments, compression detection system 102 (e.g., computing device 104 and/or processor 106) may be in combination with an insulin delivery system. Compression detection system 102 (e.g., via processor 106) may be in communication with the insulin delivery system (e.g., via wired and/or wireless means). Compression detection system 102 may transmit a signal output to the insulin delivery system, where the signal output indicates that sensor 110 is subject to compression while collecting BG measurements. The insulin delivery system may receive the signal output, where the signal output causes the insulin delivery system to perform at least one or more of initiating insulin delivery, continuing insulin delivery, disabling an alarm, and/or any combination thereof.

Steps of method 200 may be performed in various orders and sequences and are not limited to being performed in the order shown in FIG. 2. For example, second measurement data may be received prior to compression detection system 102 receiving first measurement from at least one sensor. Similarly, in some instances, compression detection system 102 may receive first measurement data from at least one sensor (e.g., sensor 110) following the detection of sensor compression. Accordingly, steps of method 200 are not limited to any particular order and may be performed on various components, whether implemented on a single computing device or multiple, distributed computing devices. Steps of method 200 may also be performed by a single sensor (e.g., sensor 100) or by multiple sensors.

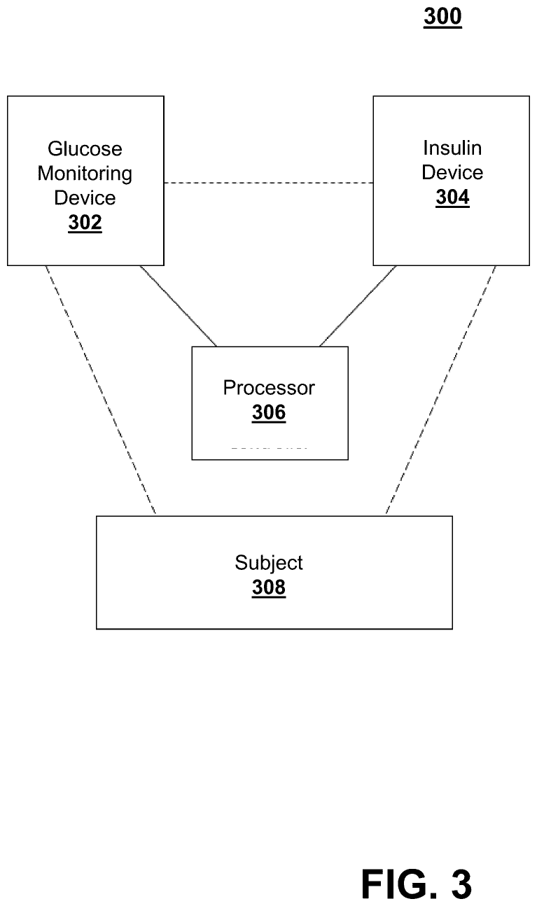

As shown in FIG. 3, embodiments may relate to an exemplary system implementation 300 for detecting sensor compression in continuous glucose monitoring. System 300 may include glucose monitoring device 302, insulin device 304, processor 306, and subject 308. In some embodiments, system 300 may include glucose monitoring device 302, processor 306, and subject 308, without insulin device 304. For example, system 300 may include glucose monitoring device 302 (e.g., and at least one sensor included therewith) in communication with at least one processor 306 to perform embodiments as disclosed herein.

In some embodiments, glucose monitoring device 302 and/or insulin device 304 may be the same as or similar to sensor 110. In some embodiments, glucose monitoring device 302 and/or insulin device 304 may include sensor 110. For example, glucose monitoring device 302 may include sensor 110 as a component of glucose monitoring device 302 or insulin device 304 may include sensor 110 as a component of insulin device 304. In some embodiments, glucose monitoring device 302, insulin device 304, and/or sensor 110 may be implemented in a single system. Alternatively, processor 306 may be implemented on a computing device separate from glucose monitoring device 302 and/or insulin device 304.

In some embodiments, glucose monitoring device 302 and/or insulin device 304 may include processor 306. For example, glucose monitoring device 302 may include processor 306 as a component of glucose monitoring device 302 or insulin device 304 may include processor 306 as a component of insulin device 304. In some embodiments, glucose monitoring device 302, insulin device 304, processor 306, and/or sensor 110 may be implemented in a single system. Alternatively, processor 306 may be implemented on a computing device separate from glucose monitoring device 302, insulin device 304, and/or sensor 110. Processor 306 may be implemented locally in glucose monitoring device 302, insulin device 304, or in a standalone device (e.g., computing device 104) (or in any combination of two or more of glucose monitoring device 302, insulin device 302, or a standalone device). In some embodiments, processor 306 may be the same as or similar to processor 106.

Glucose monitoring device 302 may include a device that may be used to monitor and/or test blood glucose levels of subject 308 (e.g., as a standalone device). Glucose monitoring device 302 may be affixed and/or attached to subject 308 to monitor blood glucose levels. Glucose monitoring device 302 may communicate (e.g., via a sensor, such as sensor 110) with subject 308 to monitor blood glucose levels of subject 308. In this way, glucose monitoring device 302 may collect measurement data (e.g., BG measurement data) for transmitting to processor 306 for detecting whether glucose monitoring device 302 and/or sensor 110 are subject to compression by subject 308. Processor 306 may execute software instructions (e.g., compression detection system 102) as a component of glucose monitoring device 302 or separate from glucose monitoring device 302. For example, processor 306 may be implemented locally in glucose monitoring device 302. In some embodiments, glucose monitoring device 302 and insulin device 304 may be implemented each as a separate device or glucose monitoring device 302 and insulin device 304 may be implemented as a single device.

In some embodiments, glucose monitoring device 302 may generate outputs, errors, parameters for accuracy improvements, and/or accuracy related information that may be transmitted, such as to processor 306, for performing various analyses, such as error analyses and/or further improvements to embodiments herein.

Insulin device 304 may include an insulin delivery system, such as an insulin pump. Insulin device 304 may communicate with subject 308 to deliver insulin to subject 308. In some embodiments, processor 306 may execute software instructions (e.g., compression detection system 102) as a component of insulin device 304 or separate from insulin device 304. For example, processor 306 may be implemented locally in insulin device 304. In some embodiments, insulin device 304 may be affixed and/or attached to subject 308 such that insulin device 304 may deliver insulin to the subject 308. Processor 306 and/or a portion of system 300 may be located remotely such that glucose monitoring device 302 and/or insulin device 304 may be operated as a telemedicine device.

Processor 306 may be implemented in hardware, software, or a combination of hardware and software. For example, processor 306 may include a common processor (e.g., a CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed with software instructions such that the processor is configured to cause the processor to perform functions when executing the software instructions. In some embodiments, processor 306 may include plural processors implemented in a single computing device (e.g., a CPU and a GPU) or processor 306 may include plural processors implemented among plural distributed computing devices. Processor 306 may be coupled to memory via a data bus to transfer data between processor 306 and memory. In some embodiments, processor 306 may be coupled to a sensor (e.g., sensor 110), glucose monitoring device 302 and/or insulin device 304 via wired (e.g., a data bus, ethernet, and/or the like) or wireless (e.g., Wi-Fi, Bluetooth, and/or the like) means and/or communication interface.

Subject 308 may include a patient located at their home or another desired location. In some embodiments, a subject may include a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

The number and arrangement of systems, hardware, and/or devices shown in FIG. 3 is provided as an example. There may be additional systems, hardware, and/or devices, fewer systems, hardware, and/or devices, different systems, hardware, and/or devices, or differently arranged systems, hardware, and/or devices than those shown in FIG. 3. Furthermore, two or more systems, hardware, and/or devices shown in FIG. 3 may be implemented within a single system, hardware, and/or device. A single system, hardware, and/or device shown in FIG. 3 may be implemented as multiple, distributed systems, hardware, and/or devices. Additionally or alternatively, a set of systems, a set of hardware, and/or a set of devices (e.g., one or more systems, one or more hardware components, one or more devices) of FIG. 3 may perform one or more functions described as being performed by another set of systems, another set of hardware, or another set of devices of FIG. 3.

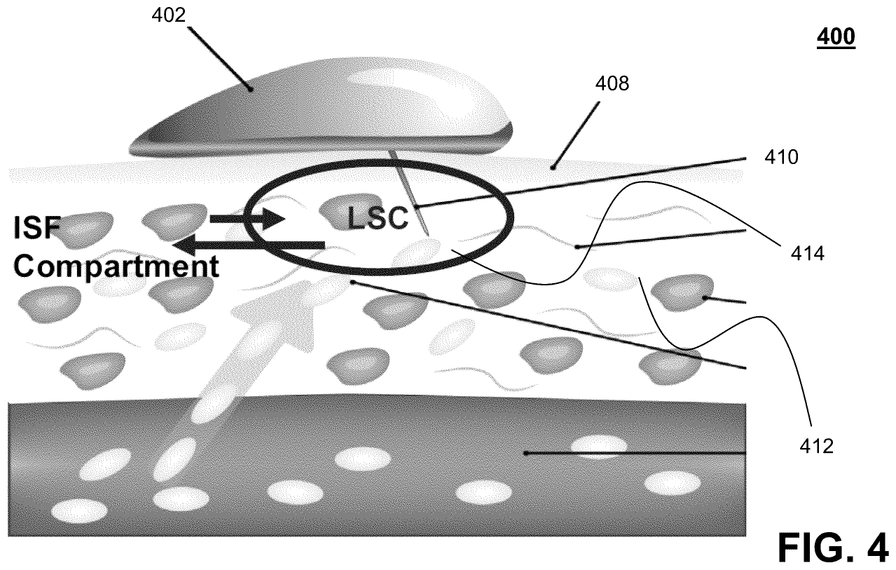

FIG. 4 shows an exemplary system 400 implementing embodiments for detecting sensor compression in CGM. For example, FIG. 4 shows example components that may be used in implementing embodiments for detecting sensor compression in CGM. where a sensor was subject to compression while collecting BG measurements. System 400 may include glucose monitoring device 402, subject 408, sensor 410, interstitial fluid compartment 412, and local sensor compartment 414.

Glucose monitoring device 402 may be the same as or similar to glucose monitoring device 302. In some embodiments, glucose monitoring device 402 and/or sensor 410 may be the same as or similar to sensor 110. In some embodiments, glucose monitoring device may include sensor 410. For example, glucose monitoring device 402 may include sensor 410 as a component of glucose monitoring device 402. In some embodiments, glucose monitoring device 402 and/or sensor 410 may be implemented in a single system such that glucose monitoring device 402 is in communication with sensor 410. In some embodiments, at least one processor (e.g., processor 106 or processor 306) may be implemented as part of glucose monitoring device 402. Alternatively, at least one processor may be implemented on a computing device separate from glucose monitoring device 402.

In some embodiments, glucose monitoring device 402, at least one processor, and/or sensor 410 may be implemented in a single system. Alternatively, at least one processor may be implemented on a computing device separate from glucose monitoring device 402, and/or sensor 410. At least one processor may be implemented locally in glucose monitoring device 402 or in a standalone device (e.g., computing device 104).

Glucose monitoring device 402 may include a device that may be used to monitor and/or test blood glucose levels (e.g., as a standalone device) of a subject (e.g., subject 408). Glucose monitoring device 402 may be affixed and/or attached to subject 408 to monitor blood glucose levels. Glucose monitoring device 402 may communicate (e.g., via a sensor, such as sensor 410) with subject 408 to monitor blood glucose levels of subject 408. In this way, glucose monitoring device 402 may collect measurement data (e.g., BG measurement data) for transmitting to at least one processor for detecting whether glucose monitoring device 402 and/or sensor 410 are subject to compression by subject 408. At least one processor (e.g., of glucose monitoring device 402, or another device) may execute software instructions (e.g., compression detection system 102) as a component of glucose monitoring device 402 or separate from glucose monitoring device 402. For example, at least one processor may be implemented locally in glucose monitoring device 402.

In some embodiments, glucose monitoring device 402 may generate outputs, errors, parameters for accuracy improvements, and/or accuracy related information that may be transmitted, such as to processor 306, for performing various analyses, such as error analyses and/or further improvements to embodiments herein.

Subject 408 may include a patient located at their home or another desired location. In some embodiments, a subject may include a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

Local sensor compartment (LSC) 414 may include tissue of subject 408 that is immediately adjacent to and/or surrounding sensor 410. LSC 414 may include an area and/or volume of tissue of subject 408 that includes an influx of glucose into LSC 414 and an outflow of glucose from LSC 414. LSC 414 may include a concentration of glucose at any given time. Interstitial fluid compartment (ISF) 412 may include tissue of subject 408 that does not immediately surround sensor 410. For example, ISF 412 may include tissue of subject 408 that excludes LSC 414. ISF 412 may include an area and/or volume tissue of subject 408 that includes an influx of glucose into ISF 412 and an outflow of glucose from ISF 412 (e.g., into LSC 414). In this way, compression detection system 102 may use an equilibrium of a concentration of glucose between LSC 414 and ISF 412 as an indicator that sensor 410 is under normal conditions and is not subject to compression. When ISF 412 and LSC 414 have concentrations of glucose that are in equilibrium, then glucose flux from LSC 414 to ISF 412 and vice versa are constant (e.g., in balance) and compression detection system 102 may determine that sensor 410 is not subject to compression (e.g., an absence of compression) based on an equilibrium of concentration of glucose between ISF 412 and LSC 414.

The number and arrangement of systems, hardware, and/or devices shown in FIG. 4 is provided as an example. There may be additional systems, hardware, and/or devices, fewer systems, hardware, and/or devices, different systems, hardware, and/or devices, or differently arranged systems, hardware, and/or devices than those shown in FIG. 4. Furthermore, two or more systems, hardware, and/or devices shown in FIG. 4 may be implemented within a single system, hardware, and/or device. A single system, hardware, and/or device shown in FIG. 4 may be implemented as multiple, distributed systems, hardware, and/or devices. Additionally or alternatively, a set of systems, a set of hardware, and/or a set of devices (e.g., one or more systems, one or more hardware components, one or more devices) of FIG. 4 may perform one or more functions described as being performed by another set of systems, another set of hardware, or another set of devices of FIG. 4.

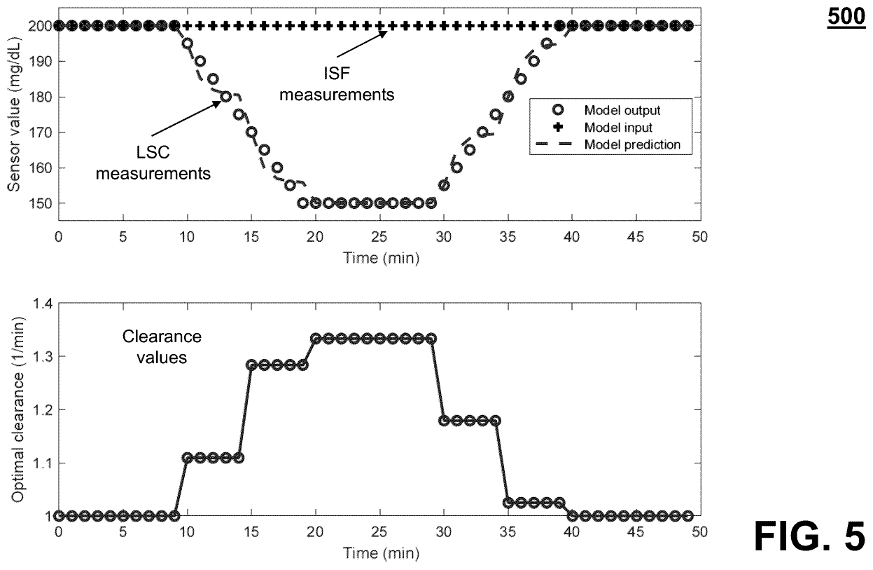

FIG. 5A shows an exemplary plot of constant CGM sensor measurements without compressions artifacts and simulated CGM measurements representing compression artifacts (top plot). FIG. 5A also shows an exemplary plot of clearance values (bottom plot) determined by a system (e.g., compression detection system 102) based on the constant CGM measurements and the simulated CGM measurements shown in the time plot of FIG. 5A. For example, the top plot shows a constant concentration of glucose in the ISF (e.g., ISF 412)(G_{ISF}) labeled as “model input” in FIG. 5A. The top plot also shows a variable concentration of glucose in the LSC (e.g., LSC 414)(G_{LSC}) labeled as “model output” in FIG. 5A. As shown in the bottom plot of FIG. 5A, the clearance values increase when a difference between G_{ISF} and G_{LSC} is present. Note that there may be at least one clearance value associated with each BG measurement in a time series of BG measurements, as shown by FIG. 5A over a 50 minute span with a plurality of BG measurements and clearance values. Embodiments also provide improvements and allow for determination of clearance values in instances where the top plot may show a variable concentration of glucose in the ISF (e.g., ISF 412)(G_{ISF}). In this way, compression detection system 102 may determine clearance values independent of a concentration of glucose in the ISF (e.g., outside of a local sensor compartment). Compression detection system 102 may determine clearance values based on differences between concentration of glucose in the ISF and concentration of glucose in the LSC. Thus, detection of sensor compression does not depend on normal physiological fluctuations that may occur in a concentration of glucose in an ISF.

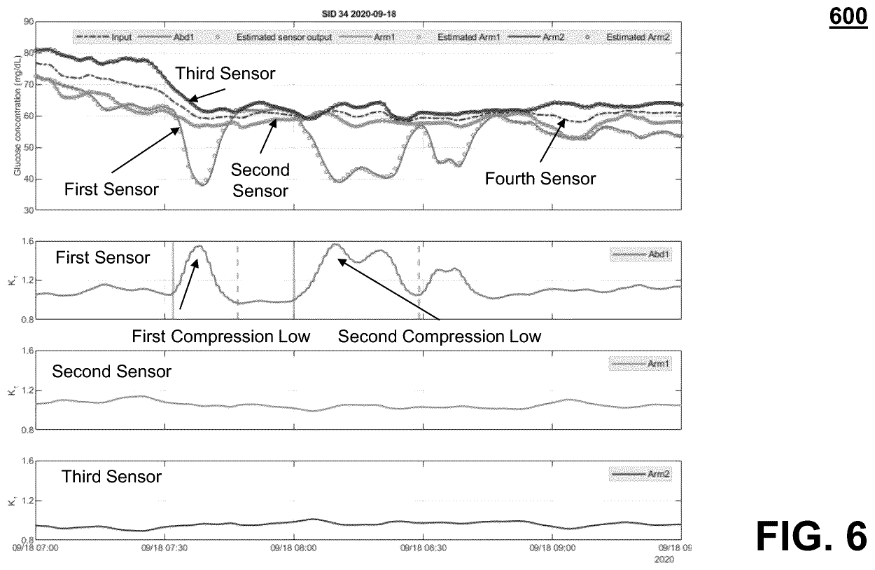

FIG. 6 shows an exemplary plot of CGM sensor measurements from a plurality of sensors along with plots of clearance values for each sensor (e.g., based on a concentration of glucose for an ISF and a concentration of glucose for a LSC) for a time series of BG measurements. For example, FIG. 6 shows embodiments provided in an environment using plural sensors to determine if any one of the plural sensors is subject to compression when collecting BG measurements. As shown in FIG. 6, plural time series of BG measurements are shown, each time series of BG measurements collected by a sensor of the plural sensors (top plot). A first time series of BG measurements collected by a first sensor is shown, having one or more compression artifacts present in the first time series of BG measurements. A second time series of BG measurements collected by a second sensor and a third time series of BG measurements collected by a third sensor are also shown. A fourth time series of BG measurements is shown representing G_{ISF} measurements collected by a fourth sensor (or plural sensors) that was not subject to compression. In FIG. 6, the fourth time series of BG measurements may include at least one BG measurement that may be used as a model input to compression detection system 102 to determine a clearance value. In this way, the fourth time series of BG measurements may be considered as a “baseline” of BG measurements collected by a sensor, while the sensor is not subject to compression.

As shown in FIG. 6, the first time series of BG measurements includes two compression lows based on the clearance values shown. Compression detection system 102 may determine clearance values for the first sensor based on the fourth time series of BG measurements (e.g., as model input) and based on the first time series of BG measurements (e.g., as model outputs). Plotting the clearance values along with the time series of BG measurements (as shown in FIG. 6) shows where compression lows occur for a sensor. The second time series of BG measurements is shown without any compression lows, as the clearance values for the second sensor are shown to be within a normal distribution (e.g., a normal range) of clearance values. The second sensor does not have any clearance value “spikes” as determined by the compression detection system 102 (and as plotted in FIG. 6) based on the fourth time series of BG measurements and the second time series of BG measurements. The third time series of BG measurements is also shown without any compression lows, as the clearance values for the third sensor are shown to be within a normal distribution of clearance values (e.g., no clearance value “spikes” in FIG. 6). It is also shown in FIG. 6 that the second and third time series of BG measurements do contain variable BG measurements are not completely constant, but the clearance values remain relatively constant. In this way, embodiments are independent of variable change in normal BG measurements (e.g., normal physiological variance). Clearance values “spike” for compression lows, allowing for accurate detection of compression in CGM sensors.

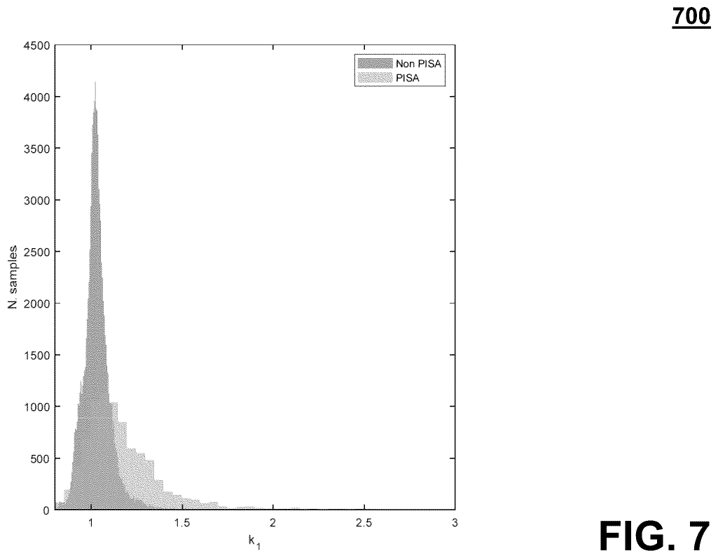

FIG. 7 shows an exemplary distribution of clearance values for a sensor under normal conditions and for a sensor subject to compression. For example, FIG. 7 shows an exemplary normal distribution of clearance values (shown in dark grey, e.g., Non PISA) and an exemplary distribution of clearance values for compression lows (shown in light grey, e.g., PISA). As shown in FIG. 7, normal clearance values (e.g., clearance values determined by compression detection system 102 for BG measurements collected by sensors that are not subject to compression) may average around 0.9-1.0, while clearance values for compression lows may average around 1.1-1.2. Such distributions of clearance values may provide sufficient differences in clearance values between normal conditions and compression-low conditions such that sensor compression may be detected by determining whether a real-time estimate of the clearance value is within the distribution of normal clearance values or whether the real-time estimate of the clearance value is within the distribution of clearance values for compression lows.

In some embodiments, compression detection system 102 may use the distribution of normal clearance values and the distribution of clearance values for compression lows to determine a predefined threshold (e.g., a clearance value threshold) that may be used for clearance values to indicate an onset of a compression low. For example, compression detection system 102 may determine a predefined threshold for clearance values where clearance values greater than the threshold indicate a sensor is subject to compression and clearance values less than the threshold indicate a sensor is not subject to compression. The predefined threshold may also be used by compression detection system 102 to indicate sensor compression and/or not sensor compression where clearance values are greater than or equal to the predefined threshold or less than or equal to the predefined threshold. In some embodiments, the predefined threshold may be equal to or between 1.05 and 1.3. In some embodiments, the predefined threshold may be equal to or between 0.9 to 1.1.

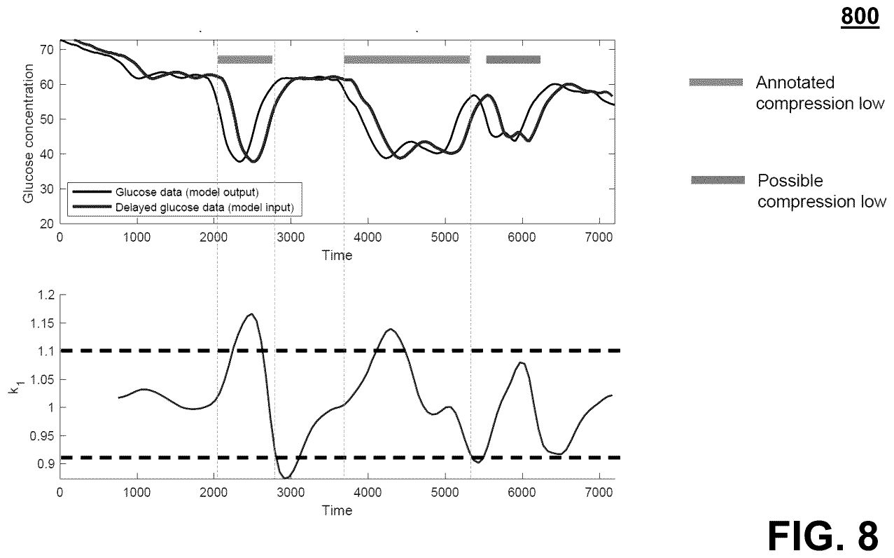

FIG. 8 shows an exemplary plot of a time series of BG measurements for a single sensor including a compression artifact detected based on clearance values for the time series of BG measurements determined using embodiments disclosed herein. FIG. 8 shows determination of clearance values using delayed measurement data collected by the single sensor (e.g., a 3 minute delay) and current measurement data (e.g., a real-time sensor trace) collected by the single sensor. The delayed measurement data collected by the single sensor may be used as input to a model (e.g., the physiological model as disclosed herein) and the current measurement data may be used as output of the model to determine clearance values for the BG measurements of the single sensor. For example, compression detection system 102 may use a model defined by:

$$\frac{d {G}_{L S C}}{d t} = - {k}_{1} * {G}_{L S C} + {k}_{0} * {G}_{ISF}$$

where G_{LSC} is a concentration of glucose in a LSC and is the output of the model, G_{ISF} is a concentration of glucose in a ISF compartment and is the input to the model, k₁ is the clearance value for a BG measurement, and k₀ is a glucose transport rate set to

$${k}_{0} = \frac{1}{min} .$$

In some min embodiments, the model may be used by compression detection system 102 at each time stamp and each BG measurement in a time series of BG measurements to determine plural clearance values. Thus, compression detection system 102 may use the model to generate a time series of clearance values, as shown in FIG. 8 (bottom plot). Using delayed measurement data collected by the single sensor is one way compression detection system 102 may determine clearance values for one sensor. In some embodiments, compression detection system 102 may determine clearance values using delayed measurement data, extrapolated measurement data, predicted measurement data (e.g., via a machine learning model), or smoothed measurement data collected from one sensor. For example, the first measurement data may include at least one BG measurement extrapolated from the at least one time series of BG measurements. In this way, compression detection system 102 may rely on measurement data collected by one sensor, where a portion of the measurement data was collected by the one sensor while the one sensor was not subject to compression. This may allow compression detection system 102 to develop a baseline of measurement data that represents measurement data not affected by compression lows.

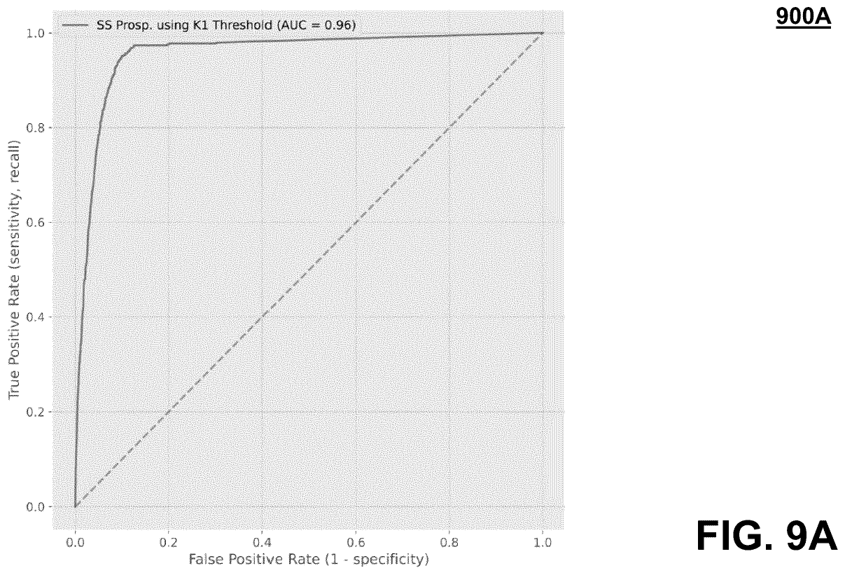

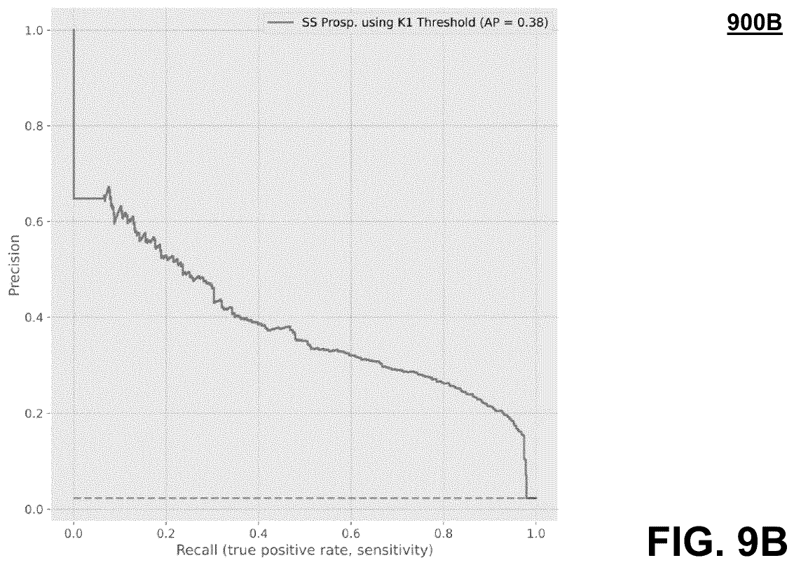

FIGS. 9A and 9B shows an exemplary plot of area under the receiver operating characteristics (ROC) curve and a precision-recall curve respectively for classifier models (e.g., a physiological model) used to classify time series of BG measurements as including a compression artifact based on clearance values to detect sensor compression. In FIG. 9A, the area under the ROC curve is 96%, indicating good performance of embodiments to accurately classify BG measurement data as indicating sensor compression using clearance values. In FIG. 9B, the precision-recall curve, which summarizes a trade-off between a true positive rate and a positive predictive value for a model, shows an average position score of 0.38. Thus, FIGS. 9A and 9B show that models of embodiments provide for accurate detection of sensor compression.

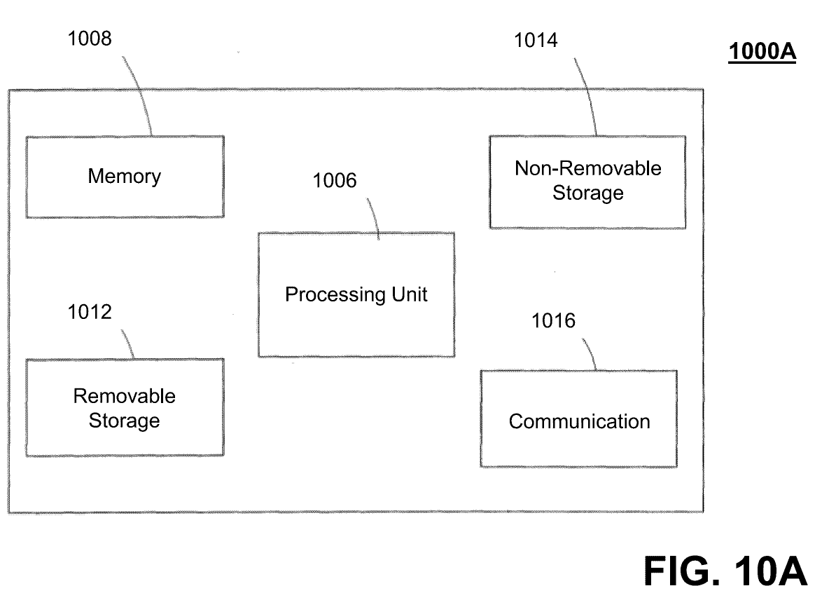

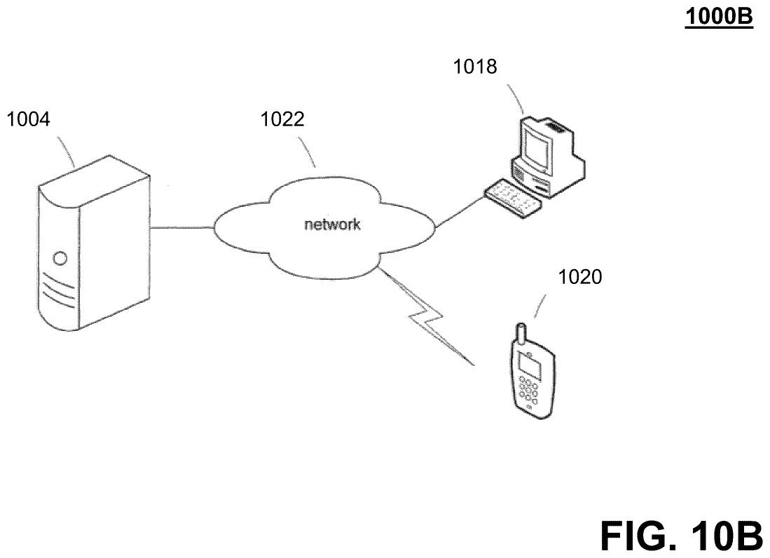

FIG. 10A shows an exemplary system configuration 1000A for an exemplary computing device (e.g., computing device 104). System configuration 1000A may include processing unit 1006, memory 1008, removable storage 1012, non-removable storage 1014, and communication interface 1016. Processing unit 1006 may be the same as or similar to processor 106 and/or processor 306. Memory 1008 may be the same as or similar to memory 108. System configuration 1000A for a computing device (e.g., computing device 104) may include at least one processing unit 1006 and memory 1008. In some embodiments, memory 1008 may include volatile (such as random access memory (RAM)), non-volatile (such as read only memory (ROM), flash memory, etc.), and/or any combination thereof.

Additionally, system configuration 1000A may include other features and/or functionality. For example, system configuration 1000A may include additional removable storage 1012 and/or non-removable storage 1014 including, but not limited to, magnetic or optical disks or tape, as well as writable electrical storage media. Computer storage media may include volatile and nonvolatile, removable and non-removable media implemented in any method or technology for storage of information such as computer readable instructions, data structures, program modules, and/or other data. Memory 1008, removable storage 1012, and non-removable storage 1014 are all examples of computer storage media. Computer storage media may include, but is not limited to, RAM, ROM, erasable programmable read only memory (EEPROM), flash memory or other memory technology compact disc read only memory (CDROM), digital versatile disks (DVD) or other optical storage, magnetic cassettes, magnetic tape, magnetic disk storage or other magnetic storage devices, or any other medium which can be used to store the desired information and which can accessed by a computing device (e.g., computing device 104). Any such computer storage media may be part of, or used in conjunction with, a computing device (e.g., computing device 104).

System configuration 1000A for an exemplary computing device may include one or more communication interfaces 1016 that allows a computing device (e.g., computing device 104) to communicate with other devices (e.g., other computing devices). Communication interface 1016 may transmit and/or carry information and/or signals in a communication media. Communication media may embody computer readable instructions, data structures, program modules and/or other data in a modulated data signal such as a carrier wave or other transport mechanism and may include any information delivery media. The term “modulated data signal” may refer to a signal that has one or more of its characteristics set or changed in such a manner as to encode, execute, and/or process information in the signal. By way of example, and not limitation, communication media may include wired media such as a wired network or direct-wired connection, and wireless media such as radio, RF, infrared and/or other wireless media. As discussed, the term computer readable media as used herein may include both storage media and/or communication media.

The number and arrangement of systems, hardware, devices and/or modules (e.g., software instructions) shown in FIG. 10A is provided as an example. There may be additional systems, hardware, devices, and/or modules, fewer systems, hardware, devices, and/or modules, different systems, hardware, devices, and/or modules, or differently arranged systems, hardware, devices, and/or modules than those shown in FIG. 10A. Furthermore, two or more systems, hardware, devices, and/or modules shown in FIG. 10A may be implemented within a single system, hardware, device, and/or module. A single system, hardware, device, and/or module shown in FIG. 10A may be implemented as multiple, distributed systems, hardware, devices, and/or modules. Additionally or alternatively, a set of systems, a set of hardware, a set of devices, and/or a set of modules (e.g., one or more systems, one or more hardware devices, one or more devices, one or more modules) of FIG. 10A may perform one or more functions described as being performed by another set of systems, another set of hardware, another set of devices, or another set of modules of FIG. 10A.

FIG. 10B shows an exemplary system environment 1000B in which systems, methods, devices, and/or computer-readable medium may be implemented. System environment 1000B may include at least one server 1004 (e.g., a network server), at least one client device 1018, a mobile device 1020, and a communication network 1022. In some embodiments, server 1004 may be the same as or similar to computing device 104. Client device 1018 may be the same as or similar to computing device 104.

FIG. 10B may include a network system including a plurality of computing devices (e.g., computing devices 104, servers 1004, and/or client devices 1018) that are in communication with a networking means (e.g., communication network 1022), such as a network with an infrastructure or an ad hoc network. The network connection may be wired connections and/or wireless connections to a plurality of computing devices. As an example, FIG. 10B shows a system environment including a network system in which embodiments may be implemented. In this example, a network system may include server 1004 (e.g. a network server), communication network 1022 (e.g. wired and/or wireless connections), client device 1018, and mobile device (e.g. a smart-phone) 1020 (or other handheld or portable device, such as a cell phone, laptop computer, tablet computer, GPS receiver, mp3 player, handheld video player, pocket projector, etc. or handheld devices (or non-portable devices) with combinations of such features). In some embodiments, it should be appreciated that server 1004, client device 1018, and/or mobile device 1020 may include a glucose monitoring device (e.g., glucose monitoring device 302). In some embodiments, it should be appreciated that server 1004, client device 1018, and/or mobile device 1020 may include a glucose monitoring device (e.g., glucose monitoring device 302), artificial pancreas, and/or an insulin device (e.g., insulin device 304), or other interventional or diagnostic device. Any of the components shown or discussed with FIG. 10B may be multiple in number.

Some embodiments may be implemented in any one of the devices of FIG. 10B. For example, execution of instructions (e.g., software instructions) or other desired processing may be performed on a same computing device that may be any one of server 1004, client device 1018, and/or mobile device 1020. Alternatively, some embodiments may be implemented and/or performed on different computing devices of the network system shown in FIG. 10B. For example, certain desired or required processing or execution may be performed on one of the computing devices of the network (e.g., server 1004, client device 1018, mobile device 1020, and/or a glucose monitoring device), whereas other processing and execution may be performed at another computing device (e.g., server 1004, client device 1018, and/or mobile device 1020) of the network system, or vice versa.

In some embodiments, certain processing and/or execution may be performed at one computing device (e.g., server 1004, client device 1018, mobile device 1020, and/or insulin device 304, artificial pancreas, or glucose monitor device 302 (or other interventional or diagnostic device)); and the other processing and/or execution (e.g., of software instructions, compression detection system 102, and/or the like) may be performed at different computing devices that may or may not be part of a network system. For example, certain processing may be performed at client device 1018, while the other processing and/or instructions are passed to server 1004 and/or mobile device 1020 where a portion of software instructions (e.g., compression detection system 102) may be executed. This scenario may be appropriate where mobile device 1020, for example, may access communication network 1022 through client device 1018 (or an access point in an ad hoc network). For another example, software instructions to be protected can be executed, encoded, and/or processed with one or more embodiments. The processed, encoded and/or executed software may then be distributed to one or more customers (e.g., client devices 1018, mobile devices 1020, and/or glucose monitoring devices 302 of customers and/or subjects). Distribution of software instructions (e.g., software modules and/or software packages) may be in a form of storage media (e.g., disk) or electronic copy.

The number and arrangement of systems, hardware, devices and/or modules (e.g., software instructions) shown in FIG. 10B is provided as an example. There may be additional systems, hardware, devices, and/or modules, fewer systems, hardware, devices, and/or modules, different systems, hardware, devices, and/or modules, or differently arranged systems, hardware, devices, and/or modules than those shown in FIG. 10B. Furthermore, two or more systems, hardware, devices, and/or modules shown in FIG. 10B may be implemented within a single system, hardware, device, and/or module. A single system, hardware, device, and/or module shown in FIG. 10B may be implemented as multiple, distributed systems, hardware, devices, and/or modules. Additionally or alternatively, a set of systems, a set of hardware, a set of devices, and/or a set of modules (e.g., one or more systems, one or more hardware devices, one or more devices, one or more modules) of FIG. 10B may perform one or more functions described as being performed by another set of systems, another set of hardware, another set of devices, or another set of modules of FIG. 10B.

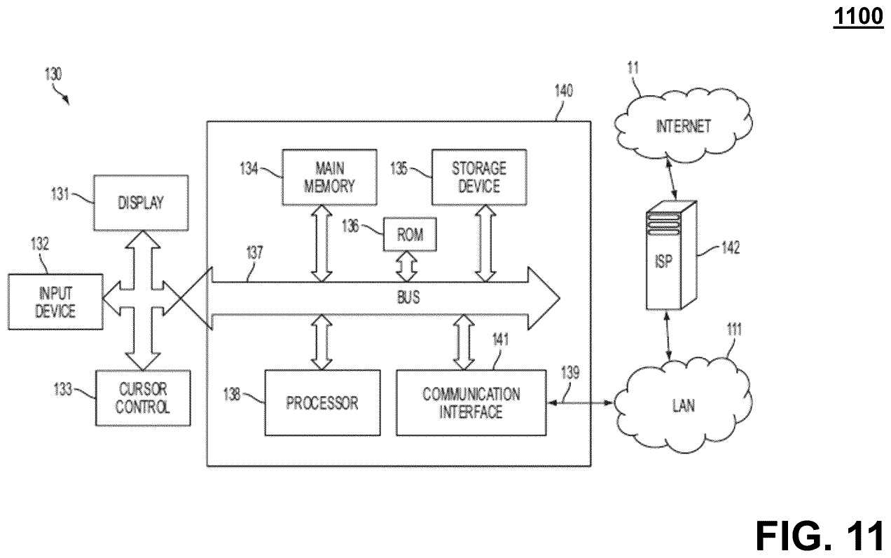

FIG. 11 is a block diagram that illustrates a system 1100 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 11. In some embodiments, computer system 140 may be the same or similar to computing device 104. The system 1100 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 11 shows various components of a computer system, it is not intended to represent any particular architecture or manner of interconnecting the components.

It will also be appreciated that network computers, handheld computers, cell phones and other data processing systems which have fewer components or perhaps more components may also be used. The computer system of FIG. 11 may, for example, be an Apple Macintosh computer or Power Book, or an IBM compatible PC. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as RAM or other dynamic storage device, coupled to bus 137 for storing information and instructions to be executed by processor 138. In some embodiments, processor 138 may be the same as or similar to processor 106 and/or processor 306.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically, computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term processor is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e.g., y), that allows the device to specify positions in a plane.

The computer system 140 may be used for implementing the methods and techniques described herein. According to one embodiment, those methods and techniques are performed by computer system 140 in response to processor 138 executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments are not limited to any specific combination of hardware circuitry and software.

The term computer-readable medium (or machine-readable medium) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that include bus 137. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 138 for execution. For example, the instructions may initially be carried on a magnetic disk of a remote computer. The remote computer can load the instructions into its dynamic memory and send the instructions over a telephone line using a modem. A modem local to computer system 140 can receive the data on the telephone line and use an infra-red transmitter to convert the data to an infra-red signal. An infra-red detector can receive the data carried in the infra-red signal and appropriate circuitry can place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137. Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Feb. 20, 2004), which is incorporated in its entirety for all purposes as if fully set forth herein.

Wireless links may also be implemented. In any such implementation, communication interface 141 sends and receives electrical, electromagnetic or optical signals that carry digital data streams representing various types of information.

Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the world wide packet data communication network Internet 11. Local network 111 and Internet 11 both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

The concept of: a) detection of CGM sensor compression (e.g., PISAs), b) improving the accuracy of CGM sensors or sensing by detecting compression artifacts inherent with devices such as medical and medicine devices and/or c) improving the action of continuous subcutaneous insulin infusion therapy and related systems, such as sensor-augmented pump (SAP), low glucose suspend (LGS), predictive low glucose suspend (PLGS), or automated insulin delivery (AID), known as an artificial pancreas are included as embodiments of the present disclosure. As provided by embodiments discussed herein, embodiments are applicable to devices for: a) providing single and/or multi-signal detection of CGM sensor compression (e.g., PISAs), b) improving the accuracy of CGM sensors or sensing by detecting compression artifacts inherent with devices such as medical and medicine devices and/or c) improving the action of continuous subcutaneous insulin infusion therapy and related systems, such as sensor-augmented pump (SAP), low glucose suspend (LGS), predictive low glucose suspend (PLGS), or automated insulin delivery (AID), known as the “artificial pancreas”, and may be implemented and utilized with the related processors, networks, computer systems, internet, and components and functions according to embodiments disclosed herein.

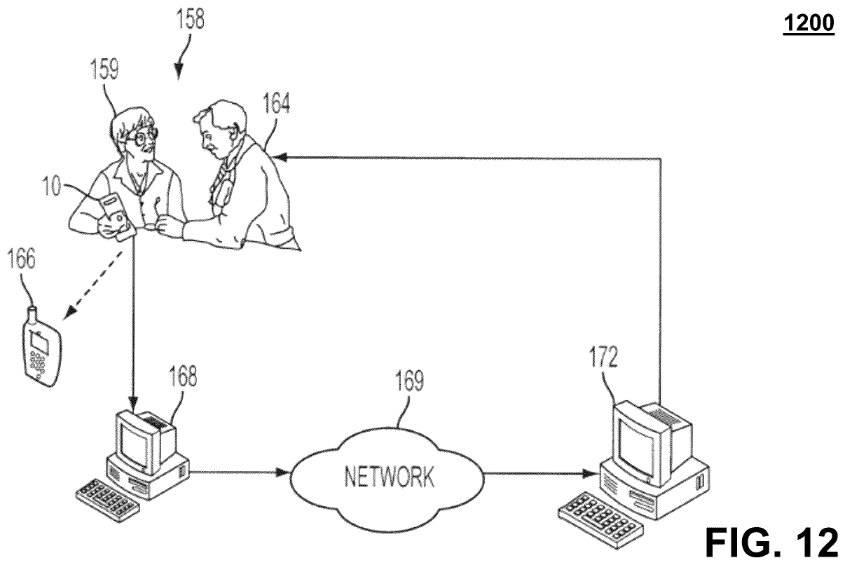

FIG. 12 shows an exemplary system and/or network in which embodiments may be implemented. In an embodiment the glucose monitor (e.g., glucose monitoring device 302), artificial pancreas or insulin device (e.g., insulin device 304) (or other interventional or diagnostic device) may be implemented by a subject (or patient) locally at home or other desired location. However, in an alternative embodiment, a glucose monitor may be implemented in a clinic setting or assistance setting. For instance, referring to FIG. 12, a clinic setup 158 provides a place for doctors (e.g. 164) or clinician/assistant to diagnose patients (e.g. 159) with diseases related with glucose and related diseases and conditions. A glucose monitoring device 10 can be used to monitor and/or test the glucose levels of the patient—as a standalone device. In some embodiments, glucose monitoring device 10 may be the same as or similar to glucose monitoring device 302.

A system or component of FIG. 12 (e.g., glucose monitoring device 10) may be affixed to the patient or in communication with the patient as desired or required. For example the system or combination of components thereof—including a glucose monitor device 10 (or other related devices or systems such as a controller, and/or an artificial pancreas, an insulin pump (or other interventional or diagnostic device), or any other desired or required devices or components)—may be in contact, communication or affixed to the patient through tape or tubing (or other medical instruments or components) or may be in communication through wired or wireless connections. Such monitor and/or test can be short term (e.g., clinical visit) or long term (e.g., clinical stay or family).

Glucose monitoring device outputs may be used by a doctor (clinician or assistant) for appropriate actions, such as insulin injection or food feeding for a patient, or other appropriate actions or modeling. Alternatively, the glucose monitoring device output can be delivered to computer terminal 168 for instant or future analyses. The delivery can be through cable or wireless or any other suitable medium. The glucose monitoring device output from the patient can also be delivered to a portable device, such as mobile device 166. The glucose monitoring device outputs with improved accuracy can be delivered to a glucose monitoring center 172 for processing and/or analyzing. Such delivery can be accomplished in many ways, such as network connection 170, which can be wired or wireless.

In addition to the glucose monitoring device outputs, errors, parameters for accuracy improvements, and any accuracy related information can be delivered, such as to computer 168, and/or glucose monitoring center 172 for performing error analyses. This can provide a centralized accuracy monitoring, modeling and/or accuracy enhancement for glucose centers (or other interventional or diagnostic centers), due to the importance of the glucose sensors (or other interventional or diagnostic sensors or devices).

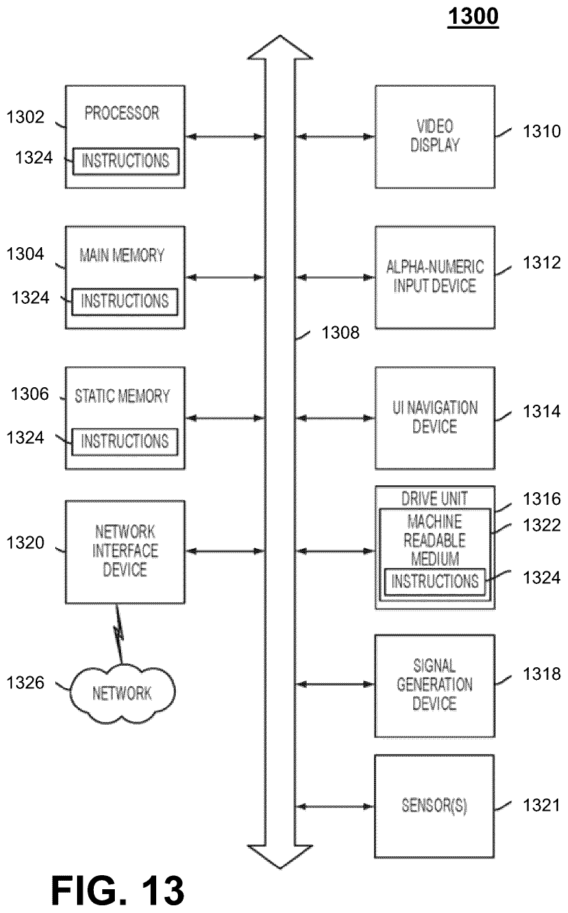

FIG. 13 is a block diagram showing an example of a machine 1300 upon which one or more aspects of embodiments may be implemented. Machine 1300 may include, but is not limited thereto, a system, method, and computer readable medium that provides for: a) multi-signal detection of CGM sensor compression (e.g., PISAs), b) improvement of the accuracy of CGM sensors or sensing by detecting compression artifacts inherent with devices such as medical and medicine devices and/or c) improvement of the action of continuous subcutaneous insulin infusion therapy and related systems, such as sensor-augmented pump (SAP), low glucose suspend (LGS), predictive low glucose suspend (PLGS), or automated insulin delivery (AID), known as the “artificial pancreas”, which illustrates a block diagram of an example machine 1300 upon which one or more embodiments (e.g., discussed methodologies) can be implemented (e.g., run).

Examples of machine 1300 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can include dedicated circuitry or logic that is specifically configured to perform one or more techniques such as discussed above, such as including a special-purpose processor, a field programmable gate array (FPGA) or an application-specific integrated circuit (ASIC). In an example, a circuit can include programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term circuit may refer to a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits may include a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can include processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a cloud computing environment or as a software as a service (SaaS) that may be executed on a remote server and accessed or used by one or more client devices. For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 1300) and software architectures that can be deployed in example embodiments.

In an example, the machine 1300 can operate as a standalone device or the machine 1300 can be connected (e.g., networked) to other machines.

In a networked deployment, the machine 1300 can operate in the capacity of either a server or a client machine in server-client network environments. In an example, machine 1300 can act as a peer machine in peer-to-peer (or other distributed) network environments. The machine 1300 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 1300. Further, while only a single machine 1300 is illustrated, the term “machine” shall also be taken to include any collection of machines that individually or jointly execute a set (or multiple sets) of instructions to perform any one or more of the methodologies discussed herein.

Example machine (e.g., computer system) 1300 can include a processor 1302 (e.g., a central processing unit (CPU), a graphics processing unit (GPU) or both), a main memory 1304 and a static memory 1306, some or all of which can communicate with each other via a bus 1308. The machine 1300 can further include a display unit 1310, an alphanumeric input device 1312 (e.g., a keyboard), and a user interface (UI) navigation device 411 (e.g., a mouse). In an example, the display unit 1310, input device 1312 and UI navigation device 1314 can be a touch screen display. The machine 1300 can additionally include a storage device (e.g., drive unit) 1316, a signal generation device 1318 (e.g., a speaker), a network interface device 1320, and one or more sensors 1321, such as a global positioning system (GPS) sensor, compass, accelerometer, or other sensor. In some embodiments, sensor 1321 may be the same as or similar to sensor 110 and/or sensor 410.

The storage device 1316 can include a machine readable medium 1322 on which is stored one or more sets of data structures or instructions 1324 (e.g., software) embodying or utilized by any one or more of the methodologies or functions described herein. The instructions 1324 can also reside, completely or at least partially, within the main memory 1304, within static memory 1306, or within the processor 1302 during execution thereof by the machine 1300. In an example, one or any combination of the processor 1302, the main memory 1304, the static memory 1306, or the storage device 1316 can constitute machine readable media.

While the machine readable medium 1322 is illustrated as a single medium, the term “machine readable medium” can include a single medium or multiple media (e.g., a centralized or distributed database, and/or associated caches and servers) that configured to store the one or more instructions 1324. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 1324 can further be transmitted or received over a communications network 1326 using a transmission medium via the network interface device 1320 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Any of the processors disclosed herein can be part of or in communication with a machine (e.g., a computer device, a logic device, a circuit, an operating module (hardware, software, and/or firmware), etc.). The processor can be hardware (e.g., processor, integrated circuit, central processing unit, microprocessor, core processor, computer device, etc.), firmware, software, etc. configured to perform operations by execution of instructions embodied in computer program code, algorithms, program logic, control, logic, data processing program logic, artificial intelligence programming, machine learning programming, artificial neural network programming, automated reasoning programming, etc. The processor can receive, process, and/or store data.

Any of the processors disclosed herein can be a scalable processor, a parallelizable processor, a multi-thread processing processor, etc. The processor can be a computer in which the processing power is selected as a function of anticipated network traffic (e.g., data flow). The processor can include an integrated circuit or other electronic device (or collection of devices) capable of performing an operation on at least one instruction, which can include a Reduced Instruction Set Core (RISC) processor, a Complex Instruction Set Computer (CISC) microprocessor, a Microcontroller Unit (MCU), a CISC-based Central Processing Unit (CPU), a Digital Signal Processor (DSP), a Graphics Processing Unit (GPU), a Field Programmable Gate Array (FPGA), etc. The hardware of such devices may be integrated onto a single substrate (e.g., silicon “die”), distributed among two or more substrates, etc. Various functional aspects of the processor may be implemented solely as software or firmware associated with the processor.

The processor can include one or more processing or operating modules. A processing or operating module can be a software or firmware operating module configured to implement any of the functions disclosed herein. The processing or operating module can be embodied as software and stored in memory, the memory being operatively associated with the processor. A processing module can be embodied as a web application, a desktop application, a console application, etc.

The processor can include or be associated with a computer or machine readable medium. The computer or machine-readable medium can include memory. Any of the memory discussed herein can be computer readable memory configured to store data. The memory can include a volatile or non-volatile, transitory or non-transitory memory, and be embodied as an in-memory, an active memory, a cloud memory, etc. Examples of memory can include flash memory, Random Access Memory (RAM), Read Only Memory (ROM), Programmable Read only Memory (PROM), Erasable Programmable Read only Memory (EPROM), Electronically Erasable Programmable Read only Memory (EEPROM), FLASH-EPROM, Compact Disc (CD)-ROM, Digital Optical Disc DVD), optical storage, optical medium, a carrier wave, magnetic cassettes, magnetic tape, magnetic disk storage or other magnetic storage devices, or any other medium which can be used to store the desired information and which can accessed by the processor.

The memory can be a non-transitory computer-readable medium. The term “computer-readable medium” (or “machine-readable medium”) as used herein is an extensible term that refers to any medium or any memory, that participates in providing instructions to the processor for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, transmission media, etc. The computer or machine readable medium can be configured to store one or more instructions or computer programs thereon. The instructions or computer programs can be in the form of algorithms, program logic, etc. that cause the processor to execute any of the functions disclosed herein.

Embodiments of the memory can include a processor module and other circuitry to allow for the transfer of data to and from the memory, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc. Communications can be via Bluetooth, near field communications, cellular communications, telemetry communications, Internet communications, etc.

Transmission of data and signals can be via transmission media. Transmission media can include coaxial cables, copper wire, fiber optics, etc. Transmission media can also take the form of acoustic or light waves, such as those generated during radio-wave and infrared data communications, or other form of propagated signals (e.g., carrier waves, digital signals, etc.).

Any of the processors can be in communication with other processors of other devices (e.g., a computer device, a computer system, a laptop computer, a desktop computer, etc.). For instance, the processor of the system configuration 100 can be in communication with a processor of another computing device 104, the processor of the computing device 104 can be in communication with a processor of a display, sensor 110, etc. Any of the processors can have transceivers or other communication devices/circuitry to facilitate transmission and reception of wireless signals. Any of the processors can include an Application Programming Interface (API) as a software intermediary that allows two or more applications to talk to each other. Use of an API can allow software of one processor to communicate with software of another processor of another device(s).

Any of the data or communication transmissions between two components can be a push operation and/or a pull operation. For instance, data transfer between the processor 106 and the memory 108 or the processor 106 and sensor 110, etc. can be push operation (e.g., the data can be pushed from the memory) and/or a pull operation (e.g., the processor can pull the data from the memory), data transfer between another system and computing device 104 can be a push and/or pull operation, etc.

When data is received by a component, it can be processed in real time, stored in memory for later processing, or some combination of both. After being processed by the component, the processed data can be used in real time, stored in memory for later use, or some combination of both. The pre-processed data and/or the processed data can be encoded, tagged, or labeled before, during, or after being stored in memory.

As noted herein, the system configuration 100 can include memory 108 containing a computer program (e.g., software instructions for compression detection system 102) that when executed can cause the processor 106 to perform any of the functions/operations disclosed herein.

The computer program can cause the processor to execute one or more machine learning models (e.g., linear regression model, tree based model, perceptron based model, Gaussian based model, etc.). It is contemplated for at least one of the machine learning models to be a random forest or AdaBoost machine learning model.

### EXAMPLES

CGM sensors widely used for the treatment of diabetes are vulnerable to the so-called “compression artifacts” or Pressure-Induced Sensor Attenuations (PISA). Compression artifacts occur frequently when a sensor is pressed, e.g. when someone sleeps on the arm where the sensor is inserted, and are characterized by a rapid drop in sensor readings, followed by eventual recovery. These deviations impact negatively various aspects of the treatment of diabetes.

We postulate that: (i) Under nominal conditions in the absence of pressure on the sensor, the interstitial fluid (ISF) where CGM sensors measure glucose and the local sensor compartment (LSC) of tissue immediately surrounding the CGM sensor needle, are in equilibrium; (ii) Pressure on the sensor results in compression of the LSC, which impairs the balance by reducing the influx of glucose, increasing glucose outflow, or both, and (iii) As a result, the electrochemical signal of the sensor is attenuated and the sensor reads low until the equilibrium is restored.

Based on this logic, in an embodiment the method and system for detection of CGM sensor compression artifacts include, but not limited thereto, three components: (i) a physiological compartmental model of glucose transport between the ISF and the LSC; (ii) a distribution of the model parameters that is observed under nominal conditions, i.e. in the absence of compression artifacts, and is thereby fixed, and (iii) a method for real-time tracking of the model parameters from CGM sensor data and detection of deviations from the nominal conditions (parameters) interpreted as compression artifacts.

An aspect of an embodiment of the method and system is intended to detect CGM sensor compression lows in real time and thereby prevent compression low effects impacting negatively the treatment of diabetes, such as false hypoglycemia alarms, or insulin shutoff by insulin delivery systems. For one of the purposes of an aspect of an embodiment of this invention, an insulin delivery system can be: (i) sensor-augmented pump (SAP) therapy; (ii) low glucose suspend (LGS) system or predictive low glucose suspend system (PLGS), or (iii) automated insulin delivery (AID), known as the “artificial pancreas.”

An aspect of an embodiment of this invention generally relates to, but not limited thereto, medicine and medical devices, as used for monitoring of blood sugar levels in the treatment of diabetes mellitus and other metabolic disorders, including but not limited to type 1 and type 2 diabetes, type 2 (T1D, T2D), latent autoimmune diabetes in adults (LADA), postprandial or reactive hyperglycemia, or insulin resistance. In alternative embodiments, the invention improves the accuracy of continuous glucose monitoring (CGM) sensors by detecting compression artifacts inherent with these devices. This improves the action of continuous subcutaneous insulin infusion therapy and related systems, such as Sensor-augmented pump (SAP), Low glucose suspend (LGS), Predictive low glucose suspend (PLGS), or Automated insulin delivery (AID), known as the “artificial pancreas.”

In alternative embodiments, an aspect of the invention provides, among other things, a physiology-based method for detection of continuous glucose monitoring (CGM) sensor artifacts referred to as “compression lows” or Pressure-Induced Sensor Attenuations (PISA). CGM sensors, widely used for the treatment of diabetes, have been vulnerable to compression low artifacts since their introduction over 20 years ago, and are still not free of this problem. Compression artifacts occur frequently when a sensor is pressed, e.g. when someone sleeps on the arm where the sensor is inserted, and are characterized by a rapid drop in sensor readings, followed by eventual recovery. These deviations impact negatively various aspects of the treatment of diabetes, including but not limited to false hypoglycemia alarms and incorrect treatment actions taken by Low glucose suspend (LGS), Predictive low glucose suspend (PLGS), or Automated insulin delivery (AID) systems.

An aspect of an embodiment of the invention is based on a physiological model of the glucose concentration at an LSC and the glucose fluxes that are occurring both during normal conditions and under pressure in the sensing area. The Compression Low Hypothesis postulates that: (1) under nominal conditions in the absence of pressure on the sensor, the interstitial fluid (ISF) where CGM sensors measure glucose and the local sensor compartment (LSC) of tissue immediately surrounding the CGM sensor needle, are in equilibrium and therefore, the glucose fluxes to and from the LSC are in balance; and (2) pressure on the sensor results in compression of the LSC, which impairs the balance by reducing the influx of glucose and possibly oxygen (not accounted for by the model), increasing glucose outflow, or both. As a result of the impaired glucose influx-outflow balance, the electrochemical signal of the sensor is attenuated and the sensor reads low until the equilibrium is restored.

A compartmental model may be used to describe the glucose diffusion phenomenon between the ISF and LSF as follows: the balance of fluxes between ISF and LSC is governed by two parameters, influx and outflow. A formal compartmental block diagram of ISF—LSC fluid exchange may be described by the following equation:

$$\frac{{dG}_{L S C}}{dt} = - {k}_{1} \cdot {G}_{L S C} + {k}_{0} \cdot {G}_{I S F} \qquad ( 1 )$$

where G_{LSC} and G_{ISF} are the glucose concentrations at the LSC and ISF respectively, k₁ is the clearance and k₀ is the glucose transport rate to the LSC, which is set to

$${k}_{0} = \frac{1}{min} .$$

The model input is a running estimate of G_{ISF} obtained at a certain frequency in different embodiments of the method, e.g. every 30 seconds if internal sensor data is used, or every minute with certain CGM devices (e.g. Abbot Libre 3), or every 5 minutes with other CGM devices (e.g. Dexcom G6, G7). The model output is the sensor reading G_{LSC}—a function of the glucose concentration in the local sensor compartment. The clearance parameter k₁ is identified using the input and the output by solving in real time the Equation (1) above. Under nominal conditions, k₁ has a stable value which increases when a compression low is experienced. As a result, the clearance parameter k₁ increases when a difference between G_{ISF} and G_{LSC} is detected. It is evident that the estimates of the clearance parameter k₁ do not depend on the trend of G_{ISF}; thus the clearance is strictly dependent on the differences between ISF and LCS glucose concentration and is independent from interstitial glucose fluctuations. This is an important design property of the Method, which ensures that compression low detection does not depend on normal physiological fluctuations of ISF glucose.

One of the key elements of an aspect of an embodiment the invention is the nominal distribution of the clearance parameter k₁. Once this is determined, deviation of the rea-time estimate of the clearance parameter k₁ from the nominal distribution indicate occurrence of a compression low. The nominal distribution of the clearance parameter k₁ was obtained using simultaneous data from several (up to 4) sensors inserted into the same individual at the same time. In this multi-sensor environment, one and exceedingly rarely two, of the sensors could experience a compression low; but, the true ISF glucose concentration is measured by the others. Thus, the model estimation proceeds as follows: (1) the model input, e.g., G_{ISF}, is obtained from the data of sensor(s) not affected by a compression low over a window of 2.5 minutes; (2) the output of the model, e.g., G_{LSC}, is obtained from the data of a sensor affected by a compression low over the same time window; (3) the Model is identified using the defined input and output signals to obtain real-time estimates of the clearance parameter k₁, thereby quantifying the outflow from the LSC; (4) the time window slides across time at small increments, e.g. 1 minute, to produce track any changes in the clearance in real time.

An example of the procedure in a multi-sensor environment is given in FIG. 6. CGM readings from three different sensors are presented (blue, black and red solid lines). Violet dashed line represents the G_{ISF} signal computed according to Step 1. In FIG. 6, Sensor Abd1 presents a sequence of two compression lows, and the corresponding identified clearance (second panel from the top) presents higher values of k₁ in the time periods with compression lows. Sensors Arm1 and Arm2 do not exhibit compression lows and the corresponding identified clearance for each one (third and fourth panels, respectively) show a constant value. In Panel B, sensor Arm2 presents compression lows flagged by increase of the clearance parameter k₁ while the other two sensors remain steady.

Using a data set of N=44 individuals wearing up to 4 sensors each and following Steps 1-4 described above, the nominal distribution of the clearance parameter k₁ and the distribution of this parameter during compression low are determined to have the following characteristics:

TABLE 1
Nominal vs. compression-low distributions of k₁

| | Non PISA | PISA |
|---|---|---|
| Mean | 1.032 | 1.213 |
| Median | 1.023 | 1.130 |
| Sd | 0.087 | 0.383 |
| CI lower | 1.031 | 1.205 |
| CI upper | 1.032 | 1.222 |
| Q2 | 0.989 | 1.035 |
| Q3 | 1.064 | 1.273 |

As seen in Table 1, these characteristics are sufficiently different between Nominal and Compression-low conditions, which allows identifying compression lows by gauging whether the real-time estimate of the clearance parameter k₁ is within normal limits or not.

Once the nominal distribution of the clearance parameter k₁ and it is apparent that it is different than the distribution of k₁ during compression-low conditions, logic of flagging a compression low in real time may be as follows: (1) in different embodiments of the method, the model input, e.g., G_{ISF}, is obtained from the data of sensor(s) not affected by a compression low over a certain time window, e.g. 1 minute, 2.5 minutes, 5 minutes, or via delay, extrapolation, prediction, or smoothing of a single sensor data using sensor work periods that are not affected by a compression low; (2) the output of the model, e.g., G_{LSC}, is the real-time data of the sensor affected or not affected by a compression low; (3) the Model is identified using the defined input and output signals to obtain real-time estimates of the clearance parameter k₁; (4) the time window slides across time at small increments, e.g. 1 minute, to produce track any changes in the clearance parameter k₁ in real time; (5) an onset of a compression low is flagged when the values of the clearance parameter k₁ exceed a certain predefined threshold corresponding to a cutoff point that differentiates well the nominal vs. compression-low distributions of k₁, e.g., about 1.1; and (6) an end of a compression low is noted when the values of the clearance parameter k₁ fall below a certain predefined threshold corresponding to a cutoff point that is below the nominal distribution of k₁, e.g. about 0.9. Step (6) may be applicable only to single-sensor solutions when the trace of the sensor is used as bot input and output to the physiological model, as described in the next section.

As an alternative to Steps (5) and (6) above, the time series of k₁ values can be used as an input to compression low detection procedures using time series forecast methods, machine learning techniques, or other approaches. Examples of this implementation are described.

A single-sensor solution in which both the input G_{ISF} and the output G_{ISF} are obtained from the data stream of the same sensor and are used to flag compression lows experience by that sensor, is the expected practical implementation of the Method. In this approach the Steps 1-6 identified above are followed and the model input G_{ISF} is obtained from a short-term (a few minutes ahead) delay, extrapolation or prediction of sensor values, e.g., using linear regression, autoregression, moving average, or other standard time-series forecast techniques.

The extrapolation/prediction horizon is determined by the frequency of data acquisition and may be typically about 5 fold larger than the time interval between consecutive data points, e.g. 2.5 to 5 minutes for a data acquisition pace of 30-60 seconds. For example, the lower panel of FIG. 8 shows an expected characteristic behavior of the clearance parameter k₁ described in Steps (5) and (6): (i) at the onset of a compression low, the delayed trace (input to the Method) remains above the sensor data and this is interpreted as increased outflow of glucose from the LSC leading to a sharp increase in the clearance parameter k₁; (ii) at the end of a compression low, the delayed trace remains below the sensor data and this is interpreted as influx of glucose into the LSC leading to decrease of the clearance parameter k₁ below its nominal limits. A third possible compression low is not flagged because the amplitude of the clearance parameter k₁ does not exceed the thresholds predefined in Steps (5) and (6).

To validate several of these embodiments, we used a Training and a Test data sets with manually annotated “reference ground truth” compression lows. There were CGM data for a total of 111 individuals, collecting 94,610 hours of sensor data: 67 subjects (60.4%) were allocated to a Training data set and 44 subjects (39.6%) were allocated to an independent Test data set. The Training data set was used to train and cross-validate the method components, while the Test data set was only used to test the Method once its components had been finalized and fixed.

Review of the time series was used to annotate each time series with compression lows whose minimum BG value was less than 85 mg/dL (e.g., hypo- or near hypo-compression lows) using the sensor data from all 111 subjects, resulting in 1,623 hypo- or near hypo-compression lows. Of the 1,623 annotations, there were 1,089 in the Training data set from, 58,403 hours of sensor data. These annotations were used as reference to test the performance of the Method in different embodiments as presented in the subsequent sections. Specifically, Steps 1-4 described in Section 4.5 above result in a time series of values of the parameter k₁, which can be used to determine the presence of compression lows in several different ways (embodiments of the method or related system).

This embodiment of the method (or related system) follows directly Steps (5) and (6) described—a certain threshold crossing for k₁ is used to determine an onset of a compression low. FIG. 9A presents a ROC performance curve in the Test set. In this case, the k₁ time series is generated using moving average; as noted, the k₁ time series can be generated using other short-term (a few minutes ahead) prediction approaches, such as a delay, extrapolation or prediction of sensor values using linear regression, autoregression, or other standard time-series forecast techniques. The area under the ROC curve is 96%, which generally signifies a very good performance. Another look at the same performance is offered by a Precision-Recall (PR) curve, which summarizes the trade-off between the true positive rate and the positive predictive value for a model in FIG. 9B. In this case, the average precision (AP) score of the PR curve is 0.38, meaning that the weighted-average precision across all thresholds, with the recalls at these thresholds used as weights, is 0.38—a result that is typically regarded as “good.” We should note that, while the area under the ROC curve and the AP score of the PR curve are related, the AP score is sometimes considered more sensitive in differentiating algorithms of similar (typically good) performance.

This embodiment of the method (or related system) follows uses the k₁ time series generated by a moving average extrapolation, as an input to two standard machine learning models: Random Forest (RF) and AdaBoost (AB). In other alternative embodiments the machine learning algorithms could be, but are not limited to, Gradient Boosted Trees, Neural Networks, Support Vector Machines, etc., or any combination of the above. These models utilize the k₁ time series in combination with other sensor internal data, e.g. temperature, but do not use sensor glucose readings as an input. In other words, the k₁ time series is the only glucose-related input to these models.

This embodiment of the method (or related system) follows uses the k₁ time series generated by a moving average extrapolation, and sensor glucose data, as an input to the same two standard machine learning models described: Random Forest and AdaBoost.

From various embodiments described, we can conclude that the Method performs well as a straightforward, single-sensor, stand-alone threshold detector of compression lows, but its performance can be improved by using advanced machine-learning models and additional inputs, such as sensor glucose. The tradeoff may be required data processing power.

It will be understood that modifications to embodiments disclosed herein can be made to meet a particular set of design criteria. For instance, any of the components discussed herein can be any suitable number or type of each to meet a particular objective. Therefore, while certain exemplary embodiments of the system and methods of making and using the same disclosed herein have been discussed and illustrated, it is to be distinctly understood that the disclosure is not limited thereto but can be otherwise variously embodied and practiced within the scope of the following claims.

It will be appreciated that some components, features, and/or configurations can be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein. Additionally, the disclosure of a range of values is a disclosure of every numerical value within that range, including the end points.

What is claimed is:

1. A system for automatically detecting sensor compression in continuous glucose monitoring in real time, the system comprising:
at least one sensor; and
at least one processor in communication with the at least one sensor, the at least one processor executing program code, wherein the at least one processor is programmed or configured to cause the processor to:
retrieve first measurement data including at least one time series of blood glucose (BG) measurements, the at least one time series being measured by the at least one sensor while not subject to compression;
receive, from the at least one sensor, second measurement data including at least one BG measurement, the at least one BG measurement measured by the at least one sensor;
determine a clearance value between BG measurements based on the first measurement data and the second measurement data; and
generate a signal output indicating that the at least one sensor is subject to compression based on the clearance value between BG measurements exceeding a predefined threshold.

2. The system of claim 1, in combination with:
at least one memory device configured to store the first measurement data, and wherein the at least one processor, as configured to retrieve the first measurement data, is programmed or configured to cause the processor to:
retrieve the first measurement data from the memory device.

3. The system of claim 1, wherein at least one time series of BG measurements of the first measurement data includes plural time stamps, each time stamp being associated with a BG measurement.

4. The system of claim 3, wherein the at least one processor, as configured to determine a clearance value between BG measurements, is programmed or configured to cause the processor to:
determine the clearance value between the at least one BG measurement of the second measurement data and a first BG measurement associated with a first time stamp of the first measurement data.

5. The system of claim 1, wherein the at least one processor, as configured to receive second measurement data including at least one BG measurement, is programmed or configured to cause the processor to:
receive, from the at least one sensor, the second measurement data including consecutive BG measurements as the BG measurements are obtained by the at least one sensor in real time.

6. The system of claim 5, wherein the at least one processor, as configured to determine a clearance value between BG measurements, is programmed or configured to cause the processor to:
determine each clearance value of plural clearance values between each consecutive BG measurement and each BG measurement of the first measurement data in real time as each consecutive BG measurement is received, a first BG measurement of the consecutive BG measurements being associated with a first time stamp of the first measurement data.

7. The system of claim 1, wherein the clearance value is determined based on:
$$\frac{d {G}_{L S C}}{d t} = - {k}_{1} * {G}_{L S C} + {k}_{0} * {G}_{ISF}$$
where, G_{LSC} is a concentration of glucose in a local sensor compartment of the at least one sensor, G_{ISF} is a concentration of glucose of interstitial fluid, dG_{LSC}/dt is a rate of change of the concentration of glucose in the local sensor compartment, k₁ is the clearance value, and k₀ is a glucose transport rate where
$${k}_{0} = \frac{1}{min} .$$

8. The system of claim 1, wherein the at least one processor, as configured to generate a signal output, is programmed or configured to cause the processor to:
indicate, in real time via outputting an indication, that the at least one sensor is subject to compression while the at least one sensor is obtaining a BG measurement.

9. The system of claim 1, in combination with:
an insulin delivery system in communication with the at least one processor, wherein the at least one processor is programmed or configured to cause the processor to:
transmit the signal output to the insulin delivery system indicating that the at least one sensor is subject to compression, wherein the signal output will cause the insulin delivery system to perform at least one or more of:
initiating insulin delivery, continuing insulin delivery, disabling an alarm, and/or any combination thereof.

10. The system of claim 3, wherein plural time stamps are separated by any one or more of 1 minute intervals, 2.5 minute intervals, and/or 5 minute intervals.

11. The system of claim 1, wherein the predefined threshold is between 1.05 and 1.3.

12. The system of claim 2, in combination with:
at least one additional sensor, wherein the at least one processor is programmed or configured to cause the processor to:
receive first measurement data including at least one time series of BG measurements from the at least one additional sensor; and
store the first measurement data in the at least one memory device.

13. The system of claim 1, wherein the at least one time series of BG measurements is measured by the at least one sensor prior to the second measurement data.

14. The system of claim 1, wherein the first measurement data includes at least one BG measurement extrapolated from the at least one time series of BG measurements.

15. A system for automatically detecting end of sensor compression in continuous glucose monitoring in real time, the system comprising:
at least one sensor; and
at least one processor in communication with the at least one sensor, the at least one processor executing program code, wherein the at least one processor is programmed or configured to cause the processor to:
receive, from the at least one sensor, first measurement data including at least one time series of blood glucose (BG) measurements, the at least one time series of BG measurements being measured by the at least one sensor while subject to compression;
receive, from the at least one sensor, second measurement data including at least one BG measurement, the at least one BG measurement being measured by the at least one sensor after the at least one time series of BG measurements has been measured by the at least one sensor;
determine a clearance value between BG measurements based on the first measurement data and the second measurement data; and
generate a signal output indicating that the at least one sensor is no longer subject to compression based on the clearance value between BG measurements being less than a predefined threshold.

16. The system of claim 15, wherein at least one time series of BG measurements of the first measurement data includes plural time stamps, each time stamp being associated with a BG measurement.

17. The system of claim 15, wherein the at least one processor, as configured to receive second measurement data including at least one BG measurement, is programmed or configured to cause the processor to:
receive, from the at least one sensor, the second measurement data including consecutive BG measurements as the BG measurements are obtained by the at least one sensor in real time.

18. The system of claim 17, wherein the at least one processor, as configured to determine a clearance value between BG measurements, is programmed or configured to cause the processor to:
determine each clearance value of plural clearance values between each consecutive BG measurement and each BG measurement of the first measurement data in real time as each consecutive BG measurement is received, a first BG measurement of the consecutive BG measurements being associated with a first time stamp of the first measurement data.

19. The system of claim 15, wherein the clearance value is determined based on:
$$\frac{d {G}_{L S C}}{d t} = - {k}_{1} * {G}_{L S C} + {k}_{0} * {G}_{ISF}$$
where, G_{LSC} is a concentration of glucose in a local sensor compartment of the at least one sensor, G_{ISF} is a concentration of glucose of interstitial fluid, dG_{LSC}/dt is a rate of change of the concentration of glucose in the local sensor compartment, k₁ is the clearance value, and k₀ is a glucose transport rate where
$${k}_{0} = \frac{1}{min} .$$

20. The system of claim 15, wherein the at least one processor, as configured to generate a signal output, is programmed or configured to cause the processor to:
output an indication in real time that the at least one sensor is not subject to compression while the at least one sensor is obtaining a BG measurement.

21. The system of claim 15, in combination with:
an insulin delivery system in communication with the at least one processor, wherein the at least one processor is programmed or configured to cause the processor to:
transmit the signal output to the insulin delivery system indicating that the at least one sensor is not subject to compression, wherein the signal output will cause the insulin delivery system to perform at least one or more of:
initiating insulin delivery, continuing insulin delivery, disabling an alarm, and/or any combination thereof.

22. The system of claim 15, wherein the predefined threshold is between 0.9 and 1.0.

23. The system of claim 15, wherein the at least one time series of BG measurements is measured by the at least one sensor prior to the second measurement data.

24. The system of claim 15, wherein the first measurement data includes at least one BG measurement extrapolated from the at least one time series of BG measurements.

25. A computer-implemented method for accurately detecting sensor compression in continuous glucose monitoring, the method comprising:
receiving first measurement data including at least one time series of blood glucose (BG) measurements measured by a first sensor not subject to compression;
receiving second measurement data including plural BG measurement measured consecutively by a second sensor subject to compression;
determining plural clearance values between BG measurements based on the first measurement data and the second measurement data; and
detecting that a third sensor is subject to compression based on a distribution of the plural clearance values.

26. The computer-implemented method of claim 25, wherein at least one time series of BG measurements of the first measurement data includes plural time stamps, each time stamp being associated with a BG measurement.

27. The computer-implemented method of claim 25, comprising:
determining the plural clearance values between the plural BG measurements of the second measurement data and the at least one time series of the first measurement data.

28. The computer-implemented method of claim 25, wherein the plural BG measurements are measured consecutively by the second sensor in real time while determining each clearance value of the plural clearance values consecutively with each BG measurement.

29. The computer-implemented method of claim 25, wherein the plural clearance values are determined based on:
$$\frac{d {G}_{L S C}}{d t} = - {k}_{1} * {G}_{L S C} + {k}_{0} * {G}_{ISF}$$
where, G_{LSC} is a concentration of glucose in a local sensor compartment of the at least one sensor, G_{ISF} is a concentration of glucose of interstitial fluid, dG_{LSC}/dt is a rate of change of the concentration of glucose in the local sensor compartment, k₁ is the clearance value, and k₀ is a glucose transport rate where
$${k}_{0} = \frac{1}{min} .$$

30. The computer-implemented method of claim 25, comprising:
indicating that the third sensor is subject to compression while the at least one sensor is obtaining a BG measurement.

31. The computer-implemented method of claim 25, comprising:
transmitting the signal output to an insulin delivery system indicating that the third sensor is subject to compression, wherein the signal output will cause the insulin delivery system to perform at least one or more of:
initiating insulin delivery, continuing insulin delivery, disabling an alarm, and/or any combination thereof.
